(19) United States
(12) Patent Application Publication
(10) Pub. No.: US 2015/0031888 A1
(43) Pub. Date: Jan. 29, 2015

(54) PROCESS FOR THE PREPARATION OF PYRIDO[2,1-a] ISOQUINOLINE DERIVATIVES BY CATALYTIC ASYMMETRIC HYDROGENATION OF AN ENAMINE

(71) Applicant: HOFFMANN-LA ROCHE INC., Nutley, NJ (US)

(72) Inventors: Stefan Abrecht, Duggingen (CH); Michelangelo Scalone, Birsfelden (CH); Rudolf Schmid, Rheinfelden (CH)

(21) Appl. No.: 14/505,946

(22) Filed: Oct. 3, 2014

Related U.S. Application Data
(63) Continuation of application No. 14/198,761, filed on Mar. 6, 2014.
Application No. 14/198,761 is a continuation of application No. 13/720,272, filed on Dec. 19, 2012, now abandoned.
Application No. 13/720,272 is a continuation of application No. 13/235,766, filed on Sep. 19, 2011, now abandoned.
Application No. 13/235,766 is a continuation of application No. 11/853,119, filed on Sep. 11, 2007, now abandoned.

(30) Foreign Application Priority Data
Sep. 15, 2006 (EP) 06120724.7

(51) Int. Cl. C07D 455/06

(52) U.S. Cl. CPC C07D 455/06
USPC 546/95

(57) ABSTRACT

The invention relates to a process for the preparation of pyrido[2,1-a] isoquinoline derivatives of the formula

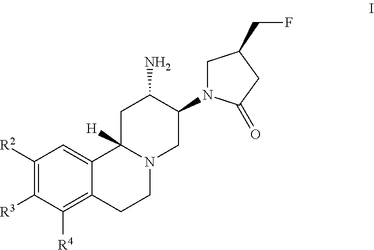

wherein R², R³ and R⁴ are as defined in the specification, comprising the steps of a) catalytic asymmetric hydrogenation of an enamine of the formula

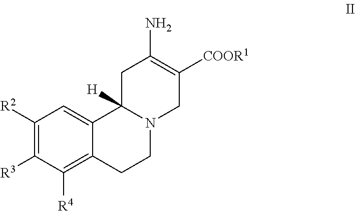

wherein R¹ is lower alkyl, in the presence of a transition metal catalyst containing a chiral diphosphane ligand, b) introduction of an amino protecting group Prot and c) amidation of the ester to form an amide of formula

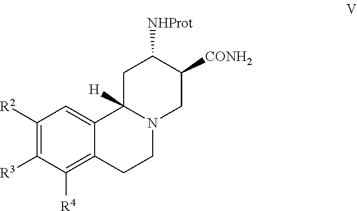

wherein R², R³, R⁴ and Prot are as defined in the specification.

### PRIORITY TO RELATED APPLICATION(S)

This application is a continuation of U.S. application Ser. No. 13/235,766 filed Sep. 19, 2011, which is a continuation of U.S. application Ser. No. 11/853,119 filed Sep. 11, 2007, which claims the benefit of European Patent Application No. 06120724.7, filed Sep. 15, 2006, which are hereby incorporated by reference in their entirety.

### FIELD OF THE INVENTION

The present invention relates to a process for the preparation of pyrido[2,1-a] isoquinoline derivatives of the formula

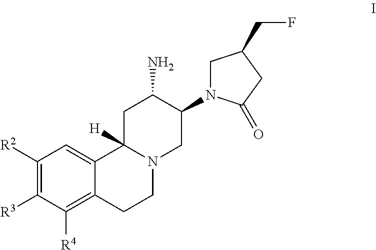

and the pharmaceutically acceptable salts thereof are useful for the treatment and/or prophylaxis of diseases which are associated with DPP IV.

All document cited or relied upon below are expressly incorporated herein by reference.

### BACKGROUND OF THE INVENTION

The pyrido[2,1-a] isoquinoline derivatives of the formula I are disclosed in PCT International Patent Appl. WO 2005/000848.

A major task in the synthesis of the compounds of formula I is the introduction of the chiral centers in the pyrido[2,1-a] isoquinoline moiety, which in the current synthesis according to the PCT Int. Appl. WO 2005/000848 involves late stage racemate separation by chiral HPLC. Such a process is however difficult to manage on technical scale. The problem to be solved was therefore to find a suitable process alternative which allows one to obtain the desired optical isomer in an earlier stage of the process, affords a higher yield and which can be conducted on technical scale.

It was found that with the process of the present invention, as outlined below, the problem could be solved.

### SUMMARY OF THE INVENTION

In an embodiment of the invention, provided is a process for the preparation of pyrido[2,1-a]isoquinoline derivatives of the formula

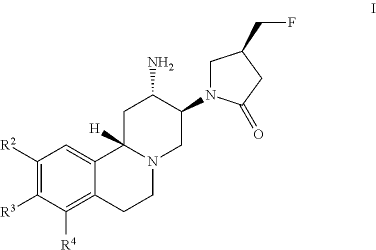

wherein R², R³ and R⁴ are each independently selected from the group consisting of hydrogen, halogen, hydroxy, lower alkyl, lower alkoxy and lower alkenyl, wherein lower alkyl, lower alkoxy and lower alkenyl may optionally be substituted by a group selected from lower alkoxycarbonyl, aryl and heterocyclyl,

comprising the steps a) and/or b) and/or c), wherein
step a) comprises catalytic asymmetric hydrogenation of an enamine of the formula

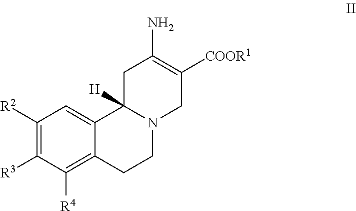

wherein R², R³ and R⁴ are as defined above and R′ is lower alkyl, in the presence of a transition metal catalyst to form the (all-S)-amino ester of formula IIIa, alone or as a mixture with 3R-epimer IIIb

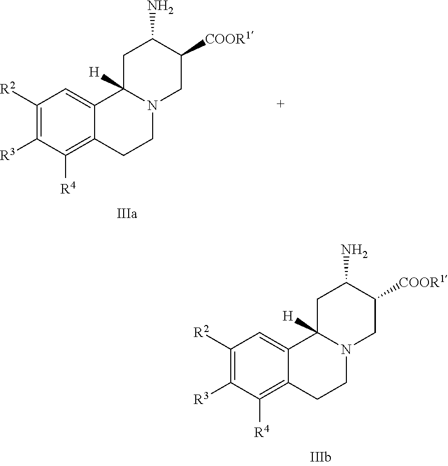

wherein R², R³ and R⁴ are as defined above and R^{1′} is lower alkyl or halogenated lower alkyl; step b) comprises the introduction of an amino protecting group Prot to form the N-protected (2S)-amino esters of formula

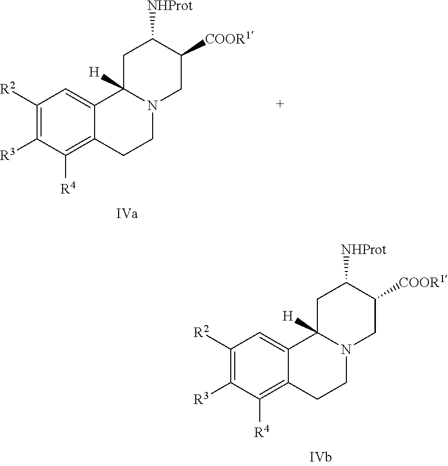

wherein R^{1′}, R², R³ and R⁴ are as defined above and Prot stands for an amino protecting group; step c) comprises amidation of the ester of formula IV to form the amide of formula

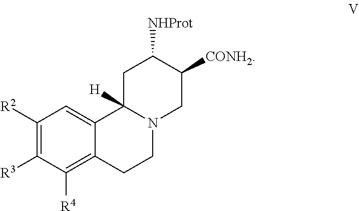

wherein R², R³, R⁴ and Prot are as defined above.

### DETAILED DESCRIPTION

Unless otherwise indicated, the following definitions are set forth to illustrate and define the meaning and scope of the various terms used to describe the invention herein.

In this specification the term “lower” is used to mean a group consisting of one to six, preferably of one to four carbon atom(s).

The term “halogen” refers to fluorine, chlorine, bromine and iodine, with fluorine, bromine and chlorine being preferred.

The term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “lower alkyl”, alone or in combination with other groups, refers to a branched or straight-chain monovalent alkyl radical of one to six carbon atoms, preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like. Preferable lower alkyl residues are methyl and ethyl, with methyl being especially preferred.

The term “halogenated lower alkyl” refers to a lower alkyl group as defined above wherein at least one of the hydrogens of the lower alkyl group is replaced by a halogen atom, preferably fluoro or chloro. Among the preferred halogenated lower alkyl groups are trifluoromethyl, difluoromethyl, fluoromethyl and chloromethyl.

The term “alkenyl” as used herein denotes an unsubstituted or substituted hydrocarbon chain radical having from 2 to 6 carbon atoms, preferably from 2 to 4 carbon atoms, and having one or two olefinic double bonds, preferably one olefinic double bond. Examples are vinyl, 1-propenyl, 2-propenyl (allyl) or 2-butenyl (crotyl).

The term “alkoxy” refers to the group R′—O—, wherein R′ is alkyl. The term “lower-alkoxy” refers to the group R′—O—, wherein R′ is a lower alkyl group as defined above. Examples of lower alkoxy groups are e.g. methoxy, ethoxy, propoxy, isopropoxy, butoxy, isobutoxy and hexyloxy, with methoxy being especially preferred.

The term “lower alkoxycarbonyl” refers to the group R′—O—C(O)—, wherein R′ is a lower alkyl group as defined above.

The term “aryl” refers to an aromatic monovalent mono- or polycarbocyclic radical, preferably phenyl or naphthyl, said aryl being unsubstituted or mono-, di- or tri-substituted, independently, by lower alkyl, lower alkoxy, halogen, cyano, azido, amino, lower dialkylamino or hydroxy. More preferably, “aryl” is unsubstituted phenyl or phenyl mono-, di- or tri-substituted, independently, by lower alkyl, lower alkoxy, halogen, cyano, azido, amino, lower dialkylamino or hydroxy.

The term “aryl¹” (as used in the definition of the diphosphine ligands) refers to an aromatic monovalent mono- or polycarbocyclic radical, preferably phenyl or naphthyl, said aryl¹ being unsubstituted or mono-, di- or tri-substituted, independently, by lower alkyl, lower alkoxy, hydroxy, halo, halogenated lower alkyl, cyano, amino, lower dialkylamino, morpholino, —SO₃H, —SO₂-lower dialkylamino, —C(O)O-lower alkyl, —C(O)-lower alkylamino, —C(O)-lower dialkylamino, phenyl and lower trialkylsilyl. Preferred “aryl¹” is phenyl, being unsubstituted or mono-, di- or tri-substituted, independently, by lower alkyl, lower alkoxy, hydroxy, halo, halogenated lower alkyl, cyano, amino, lower dialkylamino, morpholino, —SO₃H, —SO₂-lower dialkylamino, —C(O)O-lower alkyl, —C(O)-lower alkylamino, —C(O)-lower dialkylamino, phenyl and lower trialkylsilyl.

The term “lower alkylamino” refers to the group —NHR′, wherein R′ is a lower alkyl group as defined above.

The term “lower dialkylamino” refers to the group —NR′R″, wherein R′ and R″ are lower alkyl groups as defined above.

The term “cycloalkyl” refers to a monovalent carbocyclic radical of three to six, preferably four to six carbon atoms. This term is further exemplified by radicals such as cyclobutyl, cyclopentyl, cyclohexyl, cycloheptyl and cyclooctyl, with cyclopentyl and cyclohexyl being preferred. Such cycloalkyl residues may optionally be mono-, di- or tri-substituted, independently, by lower alkyl or by halogen.

The term “heterocyclyl” refers to a 5- or 6-membered aromatic or saturated N-heterocyclic residue, which may optionally contain a further nitrogen or oxygen atom, such as imidazolyl, pyrazolyl, thiazolyl, pyridyl, pyrimidyl, morpholino, piperazino, piperidino or pyrrolidino, preferably pyridyl, thiazolyl or morpholino. Such heterocyclic rings may optionally be mono-, di- or tri-substituted, independently, by lower alkyl, lower alkoxy, halo, cyano, azido, amino, lower dialkyl amino or hydroxy. Preferable substituent is lower alkyl, with methyl being preferred.

The term “heteroaryl” (as used in the definition of the diphosphine ligands) refers to a monovalent heterocyclic 5 or 6-membered aromatic radical, wherein the heteroatoms are selected from N, O or S. Preferred “heteroaryl” groups are selected from the group consisting of thienyl, indolyl, pyridinyl, pyrirnidinyl, imidazolyl, piperidinyl, furanyl, pyrrolyl, isoxazolyl, pyrazolyl, pyrazinyl, benzo[1.3]dioxolyl, benzo{b}thiophenyl and benzotriazolyl, said groups being unsubstituted or substituted by one or more substituents, independently selected from lower alkyl, lower alkoxy, halogen, halogenated lower alkyl, cyano, azido, amino, lower alkylamino, lower dialkylamino, —SO₂H, —SO₂-lower alkyl, —SO₂-lower dialkylamino, nitro, lower alkoxycarbonyl, —C(O)-lower alkylamino, —C(O)-lower dialkylamino, hydroxy, or the like.

The term “pharmaceutically acceptable salts” embraces salts of the compounds of formula I with inorganic or organic acids such as hydrochloric acid, hydrobromic acid, nitric acid, sulphuric acid, phosphoric acid, citric acid, formic acid, maleic acid, acetic acid, fumaric acid, succinic acid, tartaric acid, methanesulphonic acid, salicylic acid, p-toluenesulphonic acid and the like, which are non toxic to living organisms. Preferred salts with acids are formates, maleates, citrates, hydrochlorides, hydrobromides and methanesulfonic acid salts, with hydrochlorides being especially preferred.

In detail, the invention relates to a process for the preparation of pyrido[2,1-a] isoquinoline derivatives of the formula

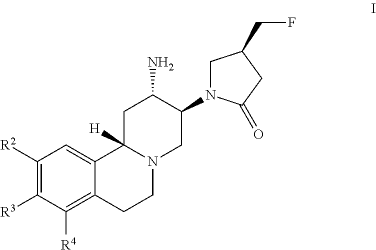

wherein R², R³ and R⁴ are each independently selected from the group consisting of hydrogen, halogen, hydroxy, lower alkyl, lower alkoxy and lower alkenyl, wherein lower alkyl, lower alkoxy and lower alkenyl may optionally be substituted by a group selected from lower alkoxycarbonyl, aryl and heterocyclyl,
comprising the steps a) and/or b) and/or c), wherein
step a) comprises catalytic asymmetric hydrogenation of an enamine of the formula

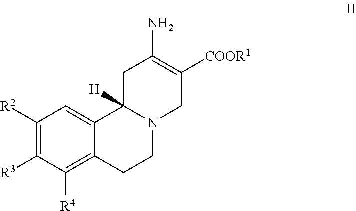

wherein R², R³ and R⁴ are as defined above and R¹ is lower alkyl, in the presence of a transition metal catalyst to form the (all-S)-amino ester of formula IIIa, alone or as a mixture with 3R-epimer IIIb

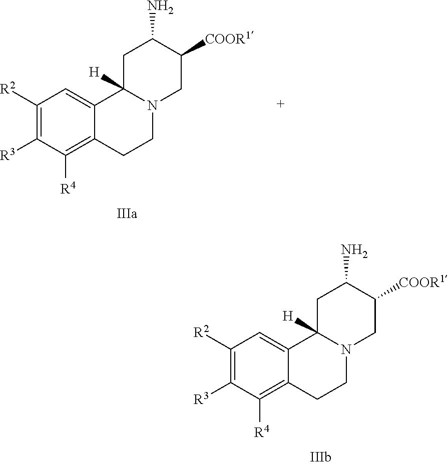

wherein R², R³ and R⁴ are as defined above and R^{1′} is lower alkyl or halogenated lower alkyl; step b) comprises the introduction of an amino protecting group Prot to form the N-protected (2S)-amino esters of formula

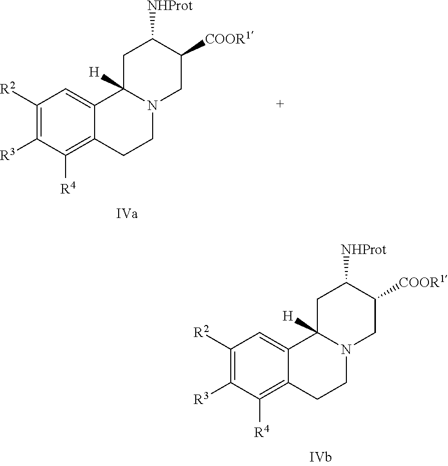

wherein R^{1′}, R², R³ and R⁴ are as defined above and Prot stands for an amino protecting group;

step c) comprises amidation of the esters of formula IVa and IVb to form the amide of formula

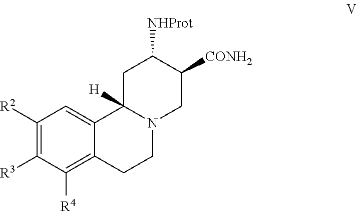

wherein R², R³, R⁴ and Prot are as defined above.

In one embodiment the process of the present invention comprises step a) as defined before.

In another embodiment the process of the present invention comprises the steps a) followed by step b) as defined before.

In yet another embodiment of the present invention the process comprises steps a) to c) together.

Step a) comprises the catalytic asymmetric hydrogenation of an enamine of the formula

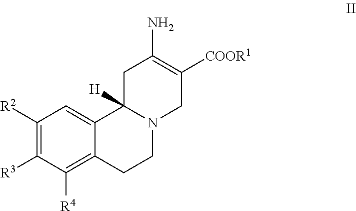

wherein R², R³ and R⁴ are each independently selected from the group consisting of hydrogen, halogen, hydroxy, lower alkyl, lower alkoxy and lower alkenyl, wherein lower alkyl, lower alkoxy and lower alkenyl may optionally be substituted by a group selected from lower alkoxycarbonyl, aryl and heterocyclyl, and R¹ is lower alkyl, in the presence of a transition metal catalyst to form the (all-S)-amino ester of formula IIIa, alone or as a mixture with 3R-epimer IIIb

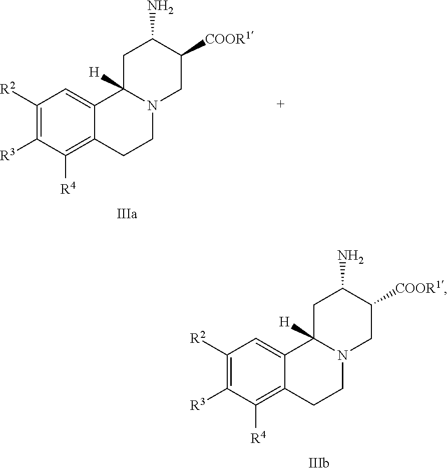

wherein R², R³ and R⁴ are as defined above and R^{1′} is lower alkyl or halogenated lower alkyl.

Depending on the solvent used in step a), transesterification of the ester group —COOR¹ is possible and thus compounds of formula IIIa and IIIB are obtained, wherein R^{1′} is lower alkyl or halogenated lower alkyl. For example, if 2,2,2-trifluoroethanol is used as solvent, compounds of formula IIIa or IIIb, wherein R^{1′} is 2,2,2-trifluoroethyl, are obtained, besides of compounds wherein R^{1′} is equal to R¹.

The enamine of formula II can be synthesized from commercially available precursors according to the scheme 1 below.

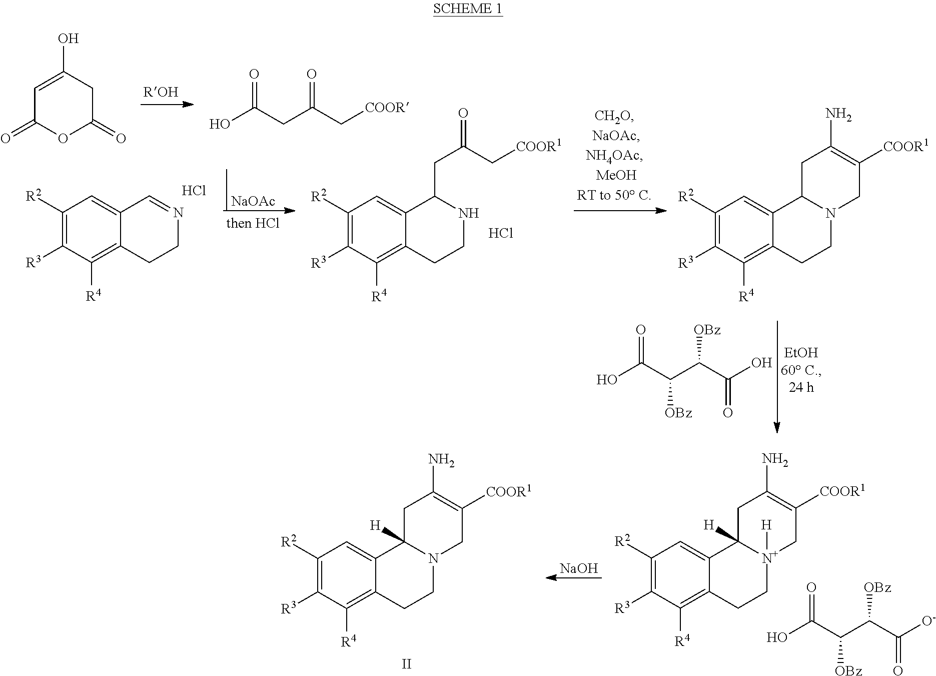

Expediently the transition metal catalyst is selected from a ruthenium, rhodium or iridium complex catalyst containing a diphosphine ligand.

Most preferably, the transition metal catalyst is a rhodium complex catalyst containing a diphosphine ligand.

In a preferred embodiment of the present invention, the diphosphine ligand is a compound selected from the group consisting of formula A to Q:

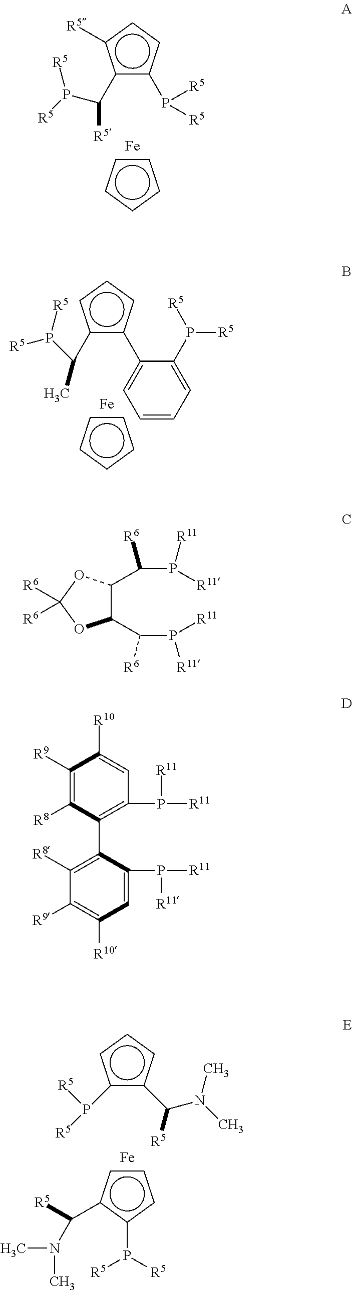

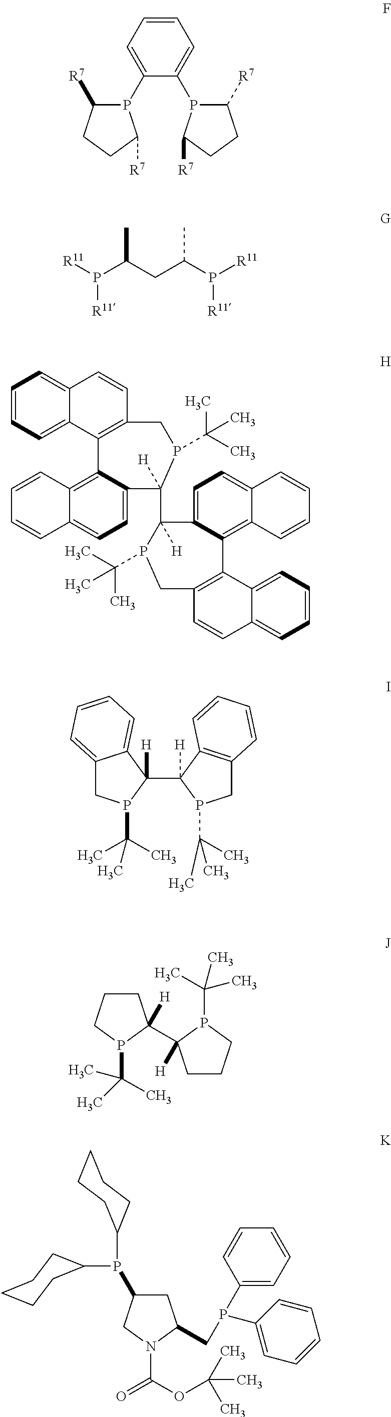

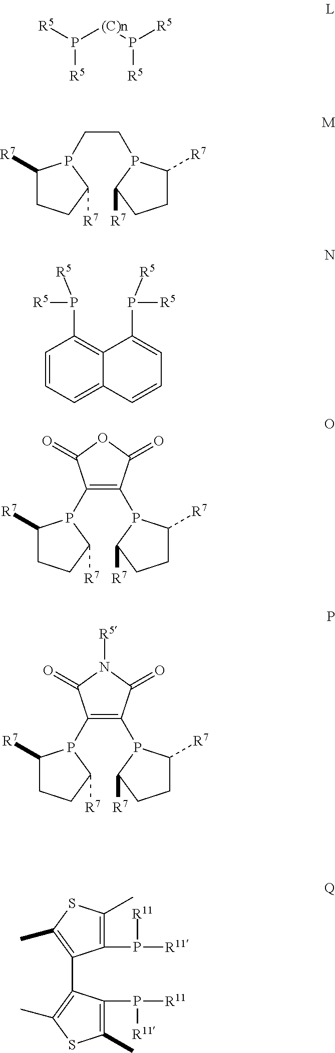

wherein
each R⁵ independently from each other is selected from the group consisting of aryl¹, heteroaryl, cycloalkyl and lower alkyl; R^{5′} is selected from the group consisting of hydrogen and lower alkyl; R^{5″} is selected from the group consisting of hydrogen, lower alkyl and phenyl;
each R⁶ independently from each other is lower alkyl;
each R⁷ independently from each other is lower alkyl or aryl¹; R⁸ and R^{8′} independently from each other are selected from the group consisting of lower alkyl, lower alkoxy, hydroxy and —O—C(O)-lower alkyl; R⁹, R^{9′}, R¹⁰ and R^{10′} independently from each other are selected from the group consisting of hydrogen, lower alkyl, lower alkoxy and lower dialkylamino; or R⁸ and R⁹, R^{8′} and R^{9′}, R⁹ and R¹⁰, R^{9′} and R^{10′} or R⁸ and R^{8′}, taken both together, are —X—(CH₂)ₙ—Y—, wherein X is —O— or —C(O)O—, Y is —O— or —N(lower alkyl)- and n is an integer from 1 to 6; or R⁸ and R⁹, R^{8′} and R^{9′}, R⁹ and R¹⁰ or R^{9′} and R^{10′}, taken both together, are a —CF₂— group, or together with the carbon atoms to which they are attached, form a naphthyl, tetrahydronaphthyl, dibenzothienyl or dibenzofuranyl ring; and R¹¹ and R^{11′} from each other is selected from the group consisting of aryl¹, lower alkyl, heteroaryl and cycloalkyl; or R¹¹ and R^{11′} together form a chiral phospholane or phosphetanc ring.

Especially preferred are diphosphine ligands of the formula

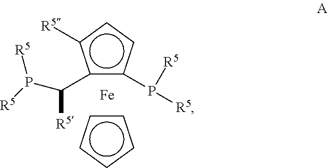

wherein each R⁵ independently from each other is selected from the group consisting of aryl¹, heterocyclyl, cycloalkyl and lower alkyl; R^{5′} is selected from the group consisting of hydrogen and lower alkyl; and R^{5″} is selected from the group consisting of hydrogen, lower alkyl and phenyl.

Preferred catalysts are selected from a rhodium complex catalyst containing a diphosphine ligand selected from the group consisting of
DCyPP, DPPP, DPPB, 1,2-Bis(iPr₂P)-acenaphthylene, PiPPP,
(S,R)—PPF—P(tBu)₂, (R)-CyMeOBIPHEP, (S,S)-MeDuphos, (R,R)-SKEWPHOS,
(1R,1′R,2S,2′S)-DuanPhos, (S,S)-BCPM, (R,R)-(Cy₂)(3,5-tBu)₂-DIOP, (R)-Cy₂-BIPHEMP,
(R)-Cy₂-MeOBIPHEP (S)-Binapine, (S,S,R)-MePHOS-MeOBIPHEP,
(R)-iPr-MeOBIPHEP, (R)-Et₂-BIPHEMP, (S,R)-Cy₂PF—PPh₂, (R,R)-l₂PPhFcCHCH₃PXyl₂,
(R,R)-Ph₂PPhFcCHCH₃PPh₂, (R,R)-Ph₂PPhFcCHCH₃PXyl₂
(S,R)-MOD-PPF—P(tBu)₂ (S)-TMBTP (all-S)-BICP (S,R)-Furyl₂PF—P(tBu)₂
(S,R)-(3,5-tBu₂-4-MeOPh)₂PF—P(tBu)₂ (S,R)-(2-MeOPh)₂PF—P(tBu)₂
(S,R)-(4-F-Ph)₂PF—P(tBu)₂ and (R)—PP(4-Ph)F—CH₂P(tBu)₂.

More preferred catalysts are selected from a rhodium or iridium complex catalyst containing a chiral diphosphine ligand selected from the group consisting of (R)-Cy₂-BIPHEMP, (R)-Cy₂-MeOBIPHEP, (S,R)-MOD-PPF—P(tBu)₂ and (S,R)-PPF—P(tBu)₂.

Especially preferred catalysts are rhodium complex catalysts containing a chiral diphosphine ligand of the formula A as defined above, most preferred is a rhodium complex catalyst containing (S,R)—PPF—P(tBu)₂ as chiral diphosphine ligand.

In the rhodium complex catalysts referred to above, rhodium is characterised by the oxidation number I. Such rhodium complexes can optionally comprise further ligands, either neutral or anionic.

Examples of such neutral ligands are e.g. olefins, e.g. ethylene, propylene, cyclooctene, 1,3-hexadiene, 1,5-hexadiene, norbornadiene (nbd bicyclo-[2.2.1]hepta-2,5-diene), (Z,Z)-1,5-cyclooctadiene (cod) or other diener which form readily soluble complexes with rhodium or ruthenium, benzene, hexamethylbenzene, 1,3,5-trimethylbenzene, p-cymene, or also solvents such as e.g. tetrahydrofiiran, dimethylformamide, acetonitrile, benzonitrile, acetone, methanol and pyridine.

Examples of such anionic ligands are halides, the group aryl-O⁻, or the group A-COO⁻, wherein A represents lower alkyl, halogenated lower alkyl and aryl. If the rhodium complex is charged, non coordinating anions such as a halide, BF₄⁻, ClO₄⁻, SbF₆⁻, AsF₆⁻, PF₆⁻, B(phenyl)₄⁻, B(3,5-di-trifluoromethyl-phenyl)₄⁻, CF₃SO₃⁻, C₆H₅SO₃⁻ are present.

Preferred catalysts comprising rhodium and a chiral diphosphine are of the formula [Rh(chiral diphosphine)LX] or [Rh(chiral diphosphine)L]⁺B⁻ wherein X is a halide such as Cl⁻, Br⁻ or I⁻, the group A-COO⁻, wherein A represents lower alkyl, aryl or halogenated lower alkyl, B is an anion of an oxyacid or a complex acid such as ClO₄⁻, PF₆⁻, BR₄⁻; wherein R is halogen or aryl, SbF₆⁻ AsF₆⁻, CF₃SO₃⁻ and C₆H₅SO₃⁻; and L is a neutral ligand as defined above. Preferably, the halide is chloride. Preferred A-COO⁻ is CH₃COO⁻ or CF₃COO⁻.

Preferred Bis CF₃SO₃⁻. If L is a ligand comprising two double bonds, e.g. 1,5-cyclooctadiene, only one such L is present. If L is a ligand comprising only one double bond, e.g. ethylene, two such L are present.

A rhodium complex catalyst can be prepared, for example, by reaction of rhodium precursors such as e.g. di-η⁴-chloro-bis[η⁴-(Z,Z)-1,5-cyclooctadiene]dirhodium(I) ([Rh(cod)Cl]₂), di-μ-chloro-bis[η⁴-norbornadiene]-dirhodium(I) ([Rh(nbd)Cl]₂), bis[η⁴-(Z,Z)-1,5-cyclooctadiene]rhodium tetra-fluoroborate ([Rh(cod)₂]BF₄) or bis[η⁴-(Z,Z)-cyclooctadiene]rhodium perchlorate ([Rh(cod)₂]ClO₄) with a chiral diphosphine ligand in a suitable inert organic or aqueous solvent (e.g. according to the method described in J. Am. Chem. Soc, 1971, 93, p. 2397-2407 or E. Jacobsen, A. Pfaltz, H. Yamamoto (Eds), Comprehensive Asymmetric Catalysis I-III, Springer Verlag Berlin (1999) and references cited therein.

In the ruthenium complex catalysts referred to above, ruthenium is characterised by the oxidation number II. Such ruthenium complexes can optionally comprise further ligands, either neutral or anionic. Examples of such neutral ligands are e.g. olefins, e.g. ethylene, propylene, cyclooctene, 1,3-hexadiene, norbornadiene, 1,5-cyclooctadiene, benzene, hexamethylbenzene, 1,3,5-trimethylbenzene, p-cymene, or also solvents such as e.g. tetrahydrofuran, dimethylformamide, acetonitrile, benzonitrile, acetone and methanol. Examples of such anionic ligands are CH₃COO⁻, CF₃COO⁻ or halides. If the ruthenium complex is charged, non coordinating anions such as halides, BF₄⁻, ClO₄⁻, SbF₆⁻, PF₆⁻, B(phenyl)₄⁻, B(3,5-di-trifluoromethyl-phenyl)₄⁻, CF₃SO₃⁻, C₆H₅SO₃⁻ are present.

Suitable ruthenium complexes in question can be represented e.g. by the following formula Ru(Z)₂D wherein Z represents halogen or the group A-COO⁻, A represents lower alkyl, aryl, halogenated lower alkyl or halogenated aryl and D represents a chiral diphosphine ligand.

These complexes can in principle be manufactured in a manner known per se, e.g. according to B. Heiser et al., Tetrahedron: Asymmetry 1991, 2, 51 or N. Feiken et al., Organometallics 1997, 16, 537 or J.-P. Genet, Acc. Chem. Res. 2003, 36, 908, M. P. Fleming et al., U.S. Pat. No. 6,545,165 B1, and references cited therein.

Conveniently and preferably, ruthenium complexes are manufactured, for example, by reacting a complex of the formula [Ru(Z¹)₂L¹ₘ]ₚ.(H₂O)₄ wherein Z¹ represents halogen or a group A¹-COO, A¹ represents lower alkyl or halogenated lower alkyl, L¹ represents a neutral ligand as defined above, m represents the number 1, 2 or 3, p represents the number 1 or 2 and q represents the number 0 or 1, with a chiral diphosphine ligand. Where m represents the number 2 or 3, the ligands can be the same or different.

Rhodium, iridium or ruthenium complex catalysts as described above can also be prepared in situ, i.e. just before use and without isolation. The solution in which such a catalyst is prepared can already contain the substrate for the enantioselective hydrogenation or the solution can be mixed with the substrate just before the hydrogenation reaction is initiated.

The asymmetric hydrogenation of a compound of formula II according to the present invention takes place at a hydrogen pressure in a range from 1 bar to 200 bar. Preferably, the asymmetric hydrogenation is carried out at a pressure of 10 to 40 bar. The reaction temperature is conveniently chosen in the range of 20° C. to 120° C. A process, wherein the asymmetric hydrogenation is carried out at a reaction temperature from 50° C. to 80° C., is preferred. This reaction can be effected in an inert organic solvent such as tetrahydrofuran, ethanol and 2,2,2-trifluoroethanol, or mixtures of 2,2,2-trifluorethanol with other solvents such as dichloromethane, methanol, ethanol, n-propanol, isopropanol, benzotrifluoride (Ph-CF₃), tetrahydrofuran, ethyl acetate or toluene. Preferably, the rhodium catalyzed hydrogenation is carried out in 2,2,2-trifluoroethanol. The ruthenium catalyzed hydrogenation is carried out in a solvent taken from the group consisting of 2,2,2-trifluoroethanol, methanol, ethanol, n-propanol and dichloromethane, or mixtures of these solvents. More preferably, the ruthenium catalyzed hydrogenation is carried out in 2,2,2-trifluoroethanol.

The amount of catalyst used in the process of the present invention is in the range of 20 to 0.005 mol % relative to substrate, preferably in the range of 1 to 0.01 mol % relative to substrate.

The process of the present invention can be carried out in the presence of an additive. Suitable additives include inorganic or organic salts and organic bases. Examples of salts are ammonium acetate, caesium carbonate, sodium formiate and sodium phosphate. Organic bases include a secondary or a tertiary amine such as for example dicyclohexylamine, diisopropylethylamine and triethylamine. Each of these bases may be used alone, or as a mixture of two or more kinds of them. The amount of base used is appropriately selected usually from the range of 0.1 to 2 equivalents, or preferably from the range of 0.1 to 0.5 equivalents to the enamine.

Step b) comprises the introduction of an amino protecting group Prot to form the N-protected (2S)-amino esters of formula

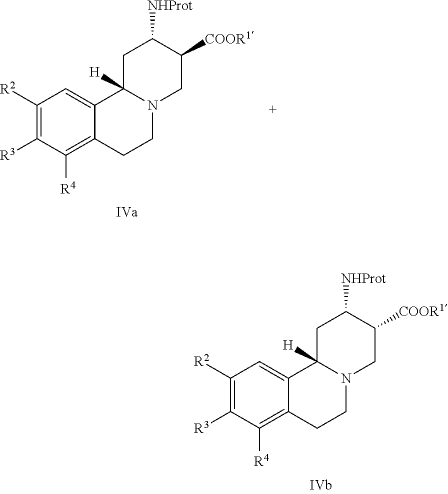

wherein R², R³ and R⁴ are as defined above, R^{1′} is lower alkyl or halogenated lower alkyl and Prot stands for an amino protecting group.

The term “amino protecting group” or “Prot” refers to any substituents conventionally used to hinder the reactivity of the amino group. Suitable amino protecting groups and its introduction are described in Green T., “Protective Groups in Organic Synthesis”, Chapter 7, John Wiley and Sons, Inc., 1991, 309-385. Suitable amino protecting groups are trichloroethoxycarbonyl, benzyloxycarbonyl (Cbz), chloroacetyl, trifluoroacetyl, phenylacetyl, formyl, acetyl, benzoyl, tert-butoxycarbonyl (Boc), para-methoxybenzyloxycarbonyl, diphenylmethoxycarbonyl, phthaloyl, succinyl, benzyl, diphenylmethyl, triphenylmethyl (trityl), methanesulfonyl, para-toluenesulfonyl, pivaloyl, trimethylsilyl, triethylsilyl, triphenylsilyl, and the like, whereby tert-butoxycarbonyl (Boc) is preferred.

Introduction of the amino protecting group can be effected following procedures well known to the skilled in the art.

Alternatively, steps a) and b) can be carried out together in one reactor without isolation of the compounds of formula IIIa or IIIb. For example, in case Prot is tert-butoxycarbonyl (Boc), the asymmetric hydrogenation of II can be carried out in the presence of Boc₂O to form directly the N-protected (S)-amino ester of formula IVa or IVb (Prot=tert-butoxycarbonyl). Preferably, a solution of Boc₂O in 2,2,2-trifluoroethanol is added continuously during the hydrogenation by pump.

In a preferred embodiment step b) comprises the manufacture of ester IV, wherein R² and R³ are methoxy, R⁴ is hydrogen and R¹ and Prot are as defined before.

Most preferably, R¹ is ethyl. Most preferably, Prot is Boc.

Step c) comprises amidation of the ester of formula IV to form the amide of formula

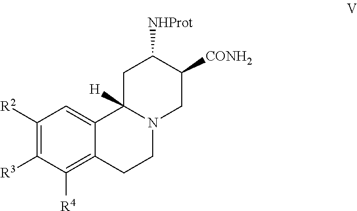

wherein R², R³, R⁴ and Prot are defined as above.

The amidation is usually performed with as suitable amidating agent, such as formamide/sodium methoxide (NaOMe), formamide/sodium ethoxide (NaOEt), acetamide/sodium methoxide and acetamide/sodium ethoxide.

The reaction can be effected in an organic solvent, such as THF, MeTHF, methanol, dimethylformamide (DMF), dioxane at temperatures of 10° C. to 70° C., preferably of 20° C. to 45° C.

In a preferred embodiment step c) comprises the manufacture of amide V wherein R² and R³ are methoxy, R⁴ is hydrogen and Prot is as defined above.

Most preferably, Prot is Boc.

The desired product is the (all-S)-diastereomer of formula V. Thus, the most preferred product is (2S,3S,11bS)-2-tert.-Butoxycarbonylamino-9,10-dimethoxy-1,3,4,6,7,11b-hexahydro-2H pyrido[2,1-a]isoquinoline-3-carboxylic acid amide having the following structure:

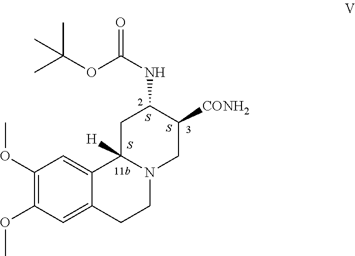

It has been found that during the amidation of the ester epimerization takes place at position 3 and thus the 3R-epimer of the formula IVb is transformed to a larger extent in the 3S-epimer of formula V.

### Further Steps:

According to still another embodiment (Scheme 2, below) the (S)-4-fluoromethyl-dihydro-furan-2-one (VII) can directly be coupled with the amino-pyrido[2,1-a]isoquinoline derivative (VI) which can be obtained from the carboxamide (V) via e.g. Hoffmann Degradation. Coupling yields the hydroxymethyl derivative of the pyrido[2,1-a] isoquinoline (VIII), which can then subsequently be cyclized to the fluoromethyl-pyrrolidin-2-one derivative (IX). The latter can be deprotected to yield the desired pyrido[2,1-a] isoquinoline derivative (I).

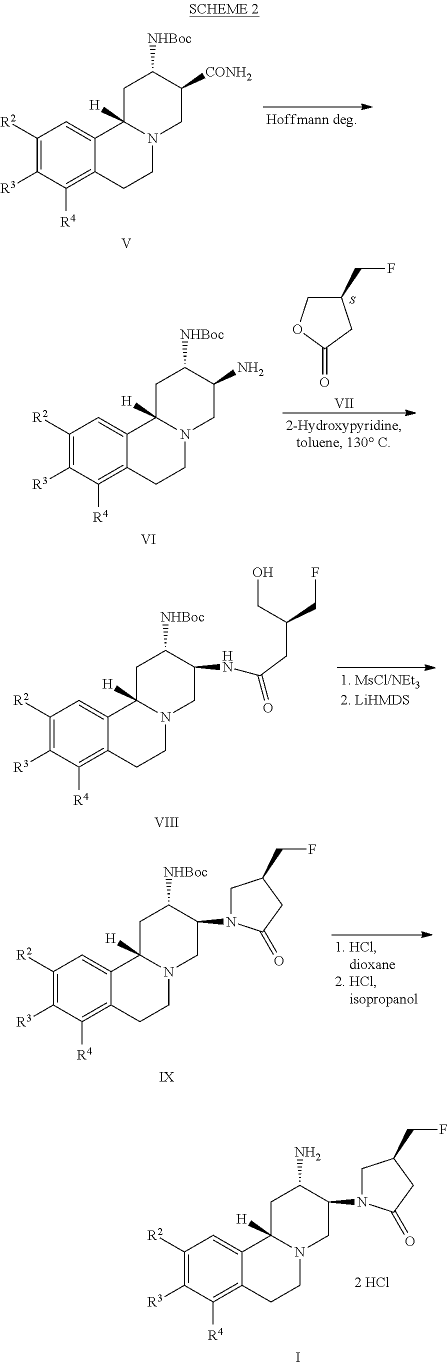

In a further preferable embodiment the process for the preparation of (S)-1-((2S,3 S,11bS)-2-amino-9,10-dimethoxy-1,3,4,6,7,11b-hexahydro-2H-pyrido[2,1-a]isoquinolin-3-yl)-4-fluoromethyl-pyrrolidin-2-one or of a pharmaceutically acceptable salt thereof comprises the subsequent steps

d) degradation of [(2S,3S,11bS)-(3-Carbamoyl-9,10-dimethoxy-1,3,4,6,7,11b-hexahydro-2H-pyrido[2,1-a]isoquinolin-2-yl)]-carbamic acid tert-butyl ester (amide of formula V wherein R² and R³ are methoxy, R⁴ is hydrogen and Prot is Boc) e) coupling of the so obtained (2S,3S,11bS)-3-amino-9,10-dimethoxy-1,3,4,6,7,11b-hexahydro-2H-pyrido[2,1-a]isoquinolin-2-yl)-carbamic acid tert-butyl ester (amine of formula VI wherein R² and R³ are methoxy, R⁴ is hydrogen and Prot is Boc) with the (S)-4-fluoromethyl-dihydro-furan-2-one of formula

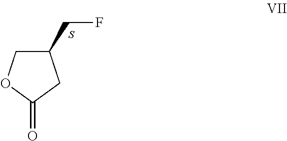

f) cyclization of the obtained (2S,3S,11bS)-3-((S)-3-fluoromethyl-4-hydroxy-butyrylamino)-9,10-dimethoxy-1,3,4,6,7,11b-hexahydro-2H-pyrido[2,1-a]isoquinolin-2-yl]-carbamic acid tert-butyl ester in the presence of a base, and
g) deprotecting the obtained (2S,3S,11Bs)-3-((4S)-fluoromethyl-2-oxo-pyrrolidin-1-yl)-9,10-dimethoxy-1,3,4,6,7,11b-hexahydro-2H-prido[2,1-a]isoquinolin-2-yl]-carbamic acid tert-butyl ester.

The pyrido[2,1-a] isoquinoline derivatives of formula (H) as disclosed in the PCT Int.

Application WO 2005/000848 are useful for the treatment and/or prophylaxis of treatment and/or prophylaxis of diseases which are associated with DPP IV such as diabetes, particularly non-insulin dependent diabetes mellitus, and/or impaired glucose tolerance, as well as other conditions wherein the amplification of action of a peptide normally inactivated by DPP-IV gives a therapeutic benefit. Surprisingly, the compounds of the present invention can also be used in the treatment and/or prophylaxis of obesity, inflammatory bowel disease, Colitis Ulcerosa, Morbus Crohn, and/or metabolic syndrome or β-cell protection. Furthermore, the compounds of the present invention can be used as diuretic agents and for the treatment and/or prophylaxis of hypertension. Unexpectedly, the compounds of the present invention exhibit improved therapeutic and pharmacological properties compared to other DPP-IV inhibitors known in the art, such as e.g. in context with pharmacokinetics and bioavailability.

The following examples shall illustrate the invention without limiting it.

### Examples

### Abbreviations

| | |
|---|---|
| DMF | N,N-Dimethylformamid |
| MeOH | Methanol |
| EtOH | Ethanol |
| TBME | Tributylmethylether |
| THF | Tetrahydrofuran |
| RT | Room Temperature |
| TFA | Trifluoracetate |
| Tf | Trifluormethansulfonate |
| TFE | 2,2,2-Trifluoroethanol |
| Boc₂O | Di-tert.-butyl-dicarbonate |

(S)-Enamine ester means (S)-2-amino-9,10-dimethoxy-1,6,7,11b-tetrahydro-4H-pyrido[2,1-a]isoquinoline-3-carboxylic acid ethyl ester (or methyl or trifluoroethyl ester if specifically indicated).

(all-S) Aminoester denotes (2S,3S,11bS)-2-Amino-9,10-dimethoxy-1,3,4,6,7,11b-hexahydro-2H pyrido[2,1-a]isoquinoline-3-carboxylic acid ethyl ((or methyl or trifluoroethyl) ester.

(all-S)—N-Boc-Ester refers to (2S,3S,11bS)-2-tert.-Butoxycarbonylamino-9,10-dimethoxy-1,3,4,6,7,11b-hexahydro-2H pyrido[2,1-a]isoquinoline-3-carboxylic acid ethyl ester; (or methyl or trifluoroethyl ester if specifically indicated).

(2R,3S,11bS)-N-Boc-Ester means (2R,3S,11bS)-2-tert.-Butoxycarbonylamino-9,10-dimethoxy-1,3,4,6,7,11b-hexahydro-2H pyrido[2,1-a]isoquinoline-3-carboxylic acid ethyl ester.

(2S,3R,11bS)-N-Boc-Ester refers to (2S,3R,11bS)-2-tert.-Butoxycarbonylamino-9,10-dimethoxy-1,3,4,6,7,11b-hexahydro-2H pyrido[2,1-a]isoquinoline-3-carboxylic acid ethyl ester.

(all-S)—N-Boc-Amide denotes (2S,3S,11bS)-2-tert.-Butoxycarbonylamino-9,10-dimethoxy-1,3,4,6,7,11b-hexahydro-2H pyrido[2,1-a]isoquinoline-3-carboxylic acid amide.

### Synthesis of Precursor Compounds

### A) Synthesis of (±)-1-(3-ethoxycarbonyl-2-oxo-propyl)-6,7-dimethoxy-1,2,3,4-tetrahydro-isoquinolinium chloride

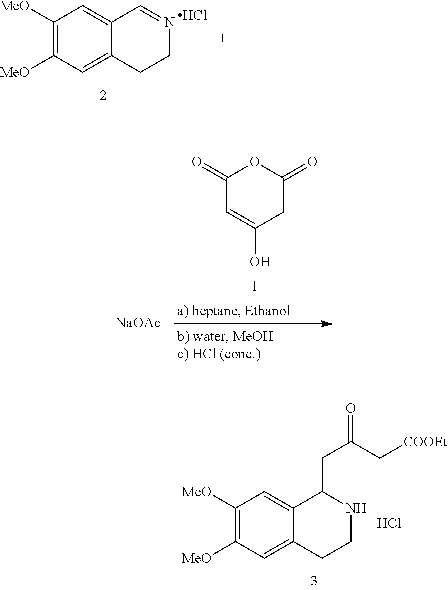 [SMILES: COc1cc2c(cc1OC)CCN=C2 ; Cl ; O=C1C=C(O)CC(=O)O1 ; CCOC(=O)CC(=O)CC1NCCc2cc(OC)c(OC)cc21 ; Cl ; "NaOAc"]

250 g of cyclic anhydride 1 was charged in the reaction vessel followed by 925 mL of heptane. 925 mL ethanol were added over 15 min to the suspension, keeping the temperature between 20-25° C. After 1 h reaction, the resulting solution was added over 1.5 h to a solution consisting of 370 g of imine hydrochloride 2, 13.33 g sodium acetate, 2.77 L ethanol and 93 mL water, keeping the temperature between 20-25° C. The product started to crystallize during the course of the reaction. After 1.5 h reaction, 16.48 mL of 37% HCl_{aq} were added followed by the addition of 2.75 L of heptane over 30 min. The yellow suspension was stirred 2 h at room temperature and filtered. The filter cake was washed with a cold (0° C.) mixture of 599 mL ethanol and 1.2 L of heptane. The crystals were dried at 50° C. under 10 mbar until constant weight to yield 534 g of amine hydrochloride 3 (88% yield, corrected for HPLC purity and residual solvent content).

The cyclic anhydride of formula 1 used as reagent was prepared as follows:

2.13 L acetic anhydride and 3 L acetic acid were charged at room temperature in the reaction vessel. The solution was cooled to 8 to 10° C. and 2 kg of 1,3-acetone dicarboxylic acid were added. The reaction mixture was stirred 3 h at 8 to 10° C. After a reaction time of about 1.5 h, a solution was almost obtained, upon which crystallization of the product started. After a reaction time of 3 h at 8 to 10° C., the suspension was filtered. The crystals were washed with 4 L toluene and dried at 45° C./10 to 20 mbar until constant weight to yield 1.33 kg of cyclic anhydride 1 (80% yield).

### B) Synthesis of (±)-2-amino-9,10-dimethoxy-1,6,7,11b-tetrahydro-4H-pyrido[2,1-a]isoquinoline-3-carboxylic acid ethyl ester

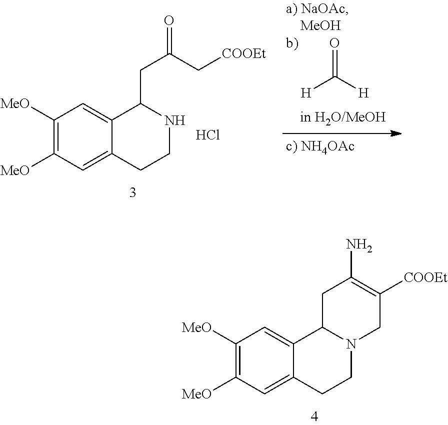 [SMILES: CCOC(=O)CC(=O)CC1NCCc2cc(OC)c(OC)cc21 ; Cl ; C=O ; CCOC(=O)C1=C(N)CC2c3cc(OC)c(OC)cc3CCN2C1]

480 g of amine hydrochloride 3 were charged in the reaction vessel followed by 7.2 L methanol and 108.9 g sodium acetate. The obtained solution was added over 25 min, keeping the temperature between 20-22° C., to a solution of 106.6 mL 36% aqueous formaldehyde in 2.4 L methanol. After 2.5 h reaction, 306.9 g ammonium acetate was added and the reaction mixture was heated to 45-50° C. After stirring overnight, the solution was concentrated to a thick oil. 4.0 L dichloromethane were added followed by 2.0 L water. 3.0 L 10% aqueous NaHCO₃ were slowly added. The organic phase was separated and washed with 3.0 L 10% aqueous NaCl. The aqueous phases were re-extracted sequentially with 3.6 L dichloromethane. The combined organic phases were concentrated and re-dissolved at reflux in 1.32 L methanol. The solution was cooled to 0° C. over 8 h, stirred 8 h at 0° C. and 5 h at −25° C., after which the suspension was filtered. The filter cake was washed in portions with in total 800 mL cold (−25° C.) methanol and 300 mL cold (−25° C.) heptane. The crystals were dried at 45° C. under 3 mbar to give 365 g enamine ester 4 (73% yield, corrected for HPLC purity and residual solvent).

### C) Synthesis of (S)-2-amino-9,10-dimethoxy-1,6,7,11b-tetrahydro-4H-pyrido[2,1-a]isoquinoline-3-carboxylic acid ethyl ester, salt with (2S,3S)-bis-benzyloxy-succinic acid

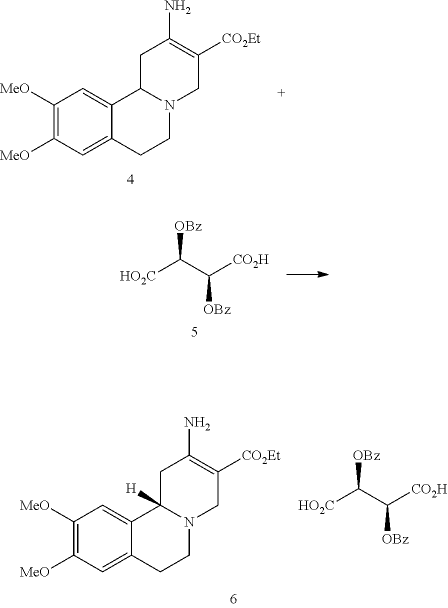

A 500-ml four-necked flask equipped with a mechanical stirrer, reflux condenser, a thermometer, and an argon in/oulet was charged with racemic enamine 4 (10.0 g, 30.1 mmol) and EtOH/H₂O 9:1 (125 ml) was added. The mixture was heated to 50° C., whereupon a clear yellowish solution was obtained. (+)-O,O′-Dibenzoyl-D-tartaric acid 5 (10.8 g, 30.1 mmol) was added in one portion to give a clear solution. After a couple of minutes, crystallization started. The mixture was allowed to slowly cool to ambient temperature over 2.5 h and was then stirred for another 14 hours. The suspension was filtered and the filter cake was washed with EtOH/H₂O (15 ml) at 0° C. After drying under vacuum, (S)-enamine salt 6 (9.37 g, 45.1% yield, 98.0% ee) was obtained as white crystals. The enantiomeric excess was determined by HPLC on chiral stationary phase using a Chiralcel OD-H column.

mp=161° C.

### D) Synthesis of (S)-2-amino-9,10-dimethoxy-1,6,7,11b-tetrahydro-4H-pyrido[2,1-a]isoquinoline-3-carboxylic acid ethyl ester

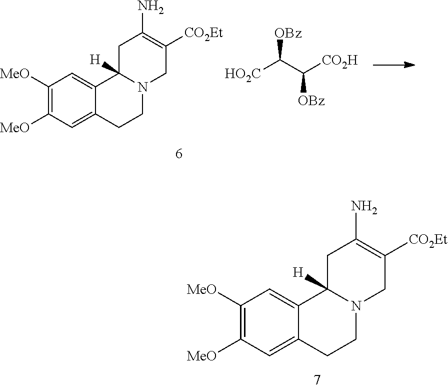

A 500-ml one-necked round bottom flask with a magnetic stirrer was charged with (S)-enamine tartaric acid salt 6 (18.6 g, 29.9 mmol, 99.0% ee) and CH₂Cl₂ (180 ml). Sodium hydroxide solution (1.0 N, 180 ml) was added and the mixture stirred at room temperature for 5 minutes. The mixture was transferred to a separating funnel and the aqueous phase was extracted with CH₂Cl₂ (180 ml). Drying over Na₂SO₄, filtration and evaporation of the solvent gave the desired (S)-enamine 7 (8.77 g, 98% yield, 99.0% ee) as a yellow foam. The enantiomeric excess was determined by HPLC on chiral stationary phase using a Chiralcel OD-H column.

### Acronyms of Diphosphine Ligands

| | |
|---|---|
| DCyPP | 1,3-Dicyclohexylphosphinopropane |
| | (commercially available from Acros Europe at Chemie |
| | Brunschwig AG, Basel, Switzerland) |
| DPPP | 1,3-Diphenylphosphinopropane |
| | (commercially available from Fluka AG, Switzerland) |
| DPPB | 1,4-Diphenylphosphinobutane |
| | (commercially available from Fluka AG, Switzerland) |
| 1,2-Bis(iPr₂P)- | 1,8-Naphthalenediylbis[bis(1-methylethyl)-phosphine |
| acenaphthylene | (preparation is described in Karacar et al, Heteroatom |
| | Chemistry 1997, 8(6), 539-550) |
| PiPPP | 1,3-Di-isopropylphosphinopropane |
| | (commercially available from Acros Europe at Chemie |
| | Brunschwig AG, Basel, Switzerland) |
| (R,S)-PPF—P(tBu)₂ | (R)-(−)-1-[(S)-2- |
| | Diphenylphosphino)ferrocenyl]ethyldi-tert.- |
| | butylphosphine ¹⁾ |
| (S,R)-PPF—P(tBu)₂ | (S)-(−)-1-[(R)-2- |
| | Diphenylphosphino)ferrocenyl]ethyldi-tert.- |
| | butylphosphine ¹⁾ |
| (R)-CyMeOBIPHEP | (R)-2,2-Bis-(dicyclohexylphosphino)-6,6-dimethoxy- |
| | 1,1′-biphenyl |
| | (preparation described in Schmid et al., Pure and |
| | Applied Chemistry 1996, 68(1), 131-8). |
| (S)-CyMeOBIPHEP | (S)-2,2-Bis-(dicyclohexylphosphino)-6,6-dimethoxy- |
| | 1,1′-biphenyl |
| | (preparation described in Schmid et al., Pure and |
| | Applied Chemistry 1996, 68(1), 131-8). |
| (R)-3,5-tBu—MeOBIPHEP | (6,6′-Dimethoxy[1,1′-biphenyl]-2,2′-diyl)bis(bis(3,5-di- |
| | tert.-butylphenyl)phosphine |
| (R,R)-MeDuphos | l,2-Bis[(2R,5R)-2,5-Dimethylphospholano]benzene |
| | (commercially available from Strem Chemicals Inc., |
| | Germany) |
| (S,S)-MeDuphos | 1,2-Bis[(2S,5S)-2,5-Dimethylphospholano]benzene |
| | commercially available from Strem Chemicals Inc., |
| | Germany) |
| (R,R)-SKEWPHOS | (2R,4R)-(−)-2,5-Dimethylphospholano]benzene |
| | (commercially available from Strem Chemicals Inc., |
| | Germany) |
| (S,S)-SKEWPHOS | (2S,4S)-(−)-2,5-Dimethylphospholano]benzene |
| | (commercially available from Strem Chemicals Inc |
| | Germany.,) |
| (1R,1′R,2S,2′S)-DuanPhos | (1R,1′R,2S,2′S)-1,1′-Bi-1H-isophosphindole,2,2′- |
| | bis(1,1-dimethylethyl)-2,2′,3,3′-tetrahydro- |
| | (commercially available from Chiral Quest Inc., USA) |
| (S,S)-BCPM | Pyrrolidinecarboxylic acid, 4-(dicyclohexylphosphino)- |
| | 2-[(diphenylphosphino)methyl]-,1,1-dimethylethyl |
| | ester, (2S-cis)- |
| | (CAS Nr 110005-30-6, preparation described in |
| | Takahashi et al. Tetrahedron Letters 1986, 27(37), |
| | 4477-80) |
| (R,R)-(Cy₂)(3,5-tBu)₂-DIOP | Bis[3,5-bis(1,1-dimethylethyl)phenyl][[(4R,5R)-5- |
| | [(dicyclohexylphosphino)methyl]-2,2-dimethyl-1,3- |
| | dioxolan-4-yl]methyl]-phosphine |
| | (prepared in analogy to Morimoto et al. Chemical & |
| | Pharmaceutical Bulletin 1993, 41(6), 1149-56) |
| (R)-Cy₂-BIPHEMP | Phosphine, dicyclohexyl[2′-(diphenylphosphino)-6,6′- |
| | dimethyl[1,1′-biphenyl]-2-yl]-,(R)-, |
| | (CAS Nr 151489-54-2, preparation described in Broger |
| | et al. PCT Int. Appl. (1993), WO 9315089 A1 and in |
| | M. Cereghetti et al, Tetrahedron Lett. 1996, 37. 5347- |
| | 50) |
| (R)-Cy₂-MeOBIPHEP | Phosphine, dicyclohexyl[2′-(diphenylphosphino)-6,6′- |
| | dimethoxy[1,1′-biphenyl]-2-yl]-,(R)-, |
| | (preparation described in Broger et al. PCT Int. Appl. |
| | (1993), WO 9315089 A1 and in M. Cereghetti et al, |
| | Tetrahedron Lett. 1996, 37. 5347-50). |
| (S)-Binapine | (3S,3′S,4S,4′S,11bS,11′bS)-(+)-4,4′-Di-t-butyl-4,4′,5,5′- |
| | tetrahydro-3,3′-bi-3H-dinaphtho[2,1-C:1′,2′- |
| | E]phosphine |
| | (commercially available from Strem Chemicals Inc., |
| | Germany) |
| (S,S,R)-MePHOS- | (2S,2′S,5S,5′S)-1,1′-[(1R)-6,6′-dimethoxy[1,1′- |
| MeOBIPHEP | biphenyl]-2,2′-diyl]bis[2,5-dimethyl-, Phospholane, |
| | (preparation is described in Schmid et al., Pure and |
| | Applied Chemistry 1996, 68(1), 131-8). |
| (R)-iPr—MeOBIPHEP | [(1R)-6,6′-dimethoxy[1,1′-biphenyl]-2,2′- |
| | diyl]bis[bis(1-methylethyl)-phosphine |
| | (preparation is described in Foricher et al. PCT Int. |
| | Appl. (1993), WO 9315091 A1) |
| (R)-Et₂-BIPHEMP | (R)-[2′-(diethylphosphino)-6,6′-dimethyl[1,1′- |
| | biphenyl]-2-yl]diphenyl-, Phosphine |
| | (preparation described in Broger et al. PCT Int. Appl. |
| | (1993), WO 9315089 A1 and in M. Cereghetti et al, |
| | Tetrahedron Lett. 1996, 37. 5347-50) |
| (R,R)-PPF—PCy₂ | (R)-1-[(R)-2-Diphenylphosphino)ferrocenyl]ethyl- |
| | dicyclohexylphosphine ¹⁾ |
| (S,R)-Cy₂PF—PPh₂ | (S)-1-[(R)-2-dicyclohexylphosphino)-ferrocenyl]ethyl- |
| | diphenylphosphine ¹⁾ |
| (R,R)-Xyl₂PPhFcCHCH₃—PXyl₂ | R)-1-[(R)-2-(2.-Di-(3,5-xylyl)-phosphinophenyl)- |
| | ferrocenyl]ethyldi(3,5-xylyl)phosphine ¹⁾ |
| (R,S)-Cy₂-PPF—P(Cy)₂ | (R)-1-[(S)-2-Dicyclohexylphosphino)ferrocenyl]- |
| | ethyldicyclohexylphosphine ¹⁾ |
| (R,R)-Ph₂PPhFCCHCH₃PPh₂ | (R)-1-[(R)-2-(2-Diphenylphosphinophenyl) |
| | ferrocenyl]-ethyldiphenylphosphine ¹⁾ |
| (R,R)-Ph₂PPhFcCHCH₃PXyl₂ | (R)-1-[(R)-2-(2-Diphenylphosphinophenyl) |
| | ferrocenyl]ethyldi-(3,5-xylyl)phosphine ¹⁾ |
| (S,R)-MOD-PPF—P(tBu)₂ | (S)-1-[(R)-2-bis-(4-methoxy-3,5-dimethylphenyl)- |
| | phosphino)ferrocenyl]ethyldi-tert.-butylphosphine ¹⁾ |
| (S)-TMBTP | (S)-2,2′,5,5′-Tetramethyl-4,4′- |
| | bis(diphenylphosphino)-3,3′-bithiophene |
| | (Commercially available from Chemi S.p. A., Via dei |
| | Lavoratori, Cinasello Balsamo, Milano 20092, Italy.) |
| (all-S)-BICP | 2,2′-bis(diphenylphosphino)-(1S,1′S,2S,2′S)-1,1′- |
| | bicyclopentyl (Commercially available from Chiral |
| | Quest Inc., Princeton Corporate Plaza, Monmouth Jet., |
| | NJ08852, USA). |
| (S,R)-Furyl₂PF—P(tBu)₂ | (S)-1-[(R)-2-(Di-2-furylphosphino)ferrocenyl]ethyldi- |
| | tert.-butylphosphine |
| (S,R)-(3,5-tBu₂-4- | (S)-1-[(R)-2-Di-(4-methoxy-3,5-di-tert.- |
| MeOPh)₂PF—P(tBu)₂ | butylphenyl)phosphino]ferrocenyl]ethyldi-tert.- |
| | butylphosphine |
| (S,R)-(2-MeOPh)₂PF—P(tBu)₂ | (S)-1-[(R)-2-Bis(2-methoxyphenyl)phosphino]- |
| | ferrocenyl]ethyldi-tert.-butylphosphine |
| (S,R)-(4-F—Ph)₂PF—P(tBu)₂ | (S)-1-[(R)-2-Bis(2-fluorophenyl)phosphino]- |
| | ferrocenyl]ethyldi-tert.-butylphosphine |
| (R)-PP(4-Ph)F—CH₂P(tBu)₂ | (R)-(4-Phenyl-2-diphenylphosphinoferrocenyl)- |
| | methyldi-tert.-butylphosphine |

¹⁾ Commercially available from Solvias AG, Basel, Switzerland.

### Example 1

### Preparation of (2S,3S,11bS)-2-tert.-Butoxycarbonylamino-9,10-dimethoxy-1,3,4,6,7,11b-hexahydro-2H pyrido[2,1-a]isoquinoline-3-carboxylic acid amide

### a) In-Situ Preparation of the Catalyst Solution

In a glove box (O₂ content<2 ppm) an Erlenmeyer flask was charged with 4.88 mg [Rh(COD)TFA]₂ (0.0075 mmol), 9.12 mg (S,R)—PPF—P(tBu)₂ (0.016 mmol) and 5 mL trifluoroethanol. The mixture was stirred for 2 h at room temperature.

### b) Asymmetric Hydrogenation (S/C 500)

In the glove box a 35 ml glass-lined autoclave equipped with a magnetic stirring bar was charged with 0.50 g (1.50 mmol) of (S)-2-amino-9,10-dimethoxy-1,6,7,11b-tetrahydro-4H-pyrido[2,1-a]isoquinoline-3-carboxylic acid ethyl ester 7, 3 ml of trifluoroethanol and 1 ml of the above catalyst solution. The autoclave was sealed and pressurized with hydrogen (30 bar). The reaction mixture was hydrogenated during 18 h at 65° C. under stirring. At this point the reaction was complete according to HPLC analysis. The hydrogenation mixture, an orange solution, was removed from the autoclave, 0.492 mg (2.26 nmol) of di-tert.-butyl-dicarbonate were added, the mixture was stirred at 40° C. for 1 h and evaporated to dryness in vacuo. HPLC analysis of the residue (0.65 g) showed a peak at RT 16.2 min (77 area %) consisting of (2S,3S,11bS)- and of (2R3S,11bS)-2-tert.-Butoxycarbonylamino-9,10-dimethoxy-1,3,4,6,7,11b-hexahydro-2H pyrido[2,1-a]isoquinoline-3-carboxylic acid ethyl ester, a peak at RT 18.2 min (13.6 area %) consisting of (2S,3S,11bS)-2-tert.-Butoxycarbonylamino-9,10-dimethoxy-1,3,4,6,7,11b-hexahydro-2H pyrido[2,1-a]isoquinoline-3-carboxylic acid trifluoroethyl ester (13.6 area %) and a peak at RT 20.3 min (1.6 area %) consisting of (2S,3R,11bS)-2-tert.-Butoxycarbonylamino-9,10-dimethoxy-1,3,4,6,7,11b-hexahydro-2H pyrido[2,1-a]isoquinoline-3-carboxylic acid ethyl ester.

### c) Amidation

A solution of the above residue in 7 ml of THF was treated with 0.60 ml of formamide (15.1 mmol) and 0.84 ml of a 30% solution of sodium methylate in methanol (4.5 mmol) and stirred at room temperature over night. To the resulting suspension was added 3.5 ml of water, the mixture was heated at reflux for 3 h, cooled to room temperature and filtered with suction. The filter cake was washed with a total of 6 ml of water/THF 1:2, with 2 ml of deionized water and dried at 60° C. at 5 mbar for 5 h to afford 0.46 g of (2S,3S,11bS)-N-Boc-Amide 8 with 99.1 area % purity by HPLC.

HPLC conditions for determination of conversion and selectivity of hydrogenation and amidation: Agilent Mod. 1100 with X-Bridge C18 column (Waters, Taunton, Mass., USA), 3.5 μm pores, 4.6×150 mm; eluent: A (H₂O with 5% acetonitrile and 1% triethylamine), B (acetonitrile with 1% triethylamine). Program: start 85% A/15% B for 2 min, then to 30% A/70% B within 18 min, 10 min isocratic, wavelength 285 nm.

Elemental analysis for C₂₁H₃₁N₃O₅:

C, 62.20 (calc. 62.10); H, 7.71 (calc. 7.63), N, 10.36 (calc. 10.28)

### Example 2

### a) In-Situ Preparation of the Catalyst Solution

In a glove box (O₂ content<2 ppm) an Erlenmeyer flask was charged with 1.95 mg [Rh(COD)TFA]₂ (0.0030 mmol), 2.89 mg DCyPP (0.0066 mmol) and 1 mL trifluoroethanol. The mixture was stirred for 2 h at room temperature.

### b) Asymmetric Hydrogenation (S/C 25)

In the glove box the above catalyst solution was added in a glass vial to 0.050 g (0.15 mmol) of (S)-2-amino-9,10-dimethoxy-1,6,7,11b-tetrahydro-4H-pyrido[2,1-a]isoquinoline-3-carboxylic acid ethyl ester 7 and the vial was placed in an autoclave. The autoclave was sealed and pressurized with hydrogen (30 bar). The reaction mixture was hydrogenated during 18 h at 50° C. under stirring. The hydrogenation mixture was removed from the autoclave, 0.050 mg (0.23 mmol) of di-tert.-butyl-dicarbonate were added, the mixture was stirred at 40° C. for 1 h and evaporated to dryness in vacuo. HPLC analysis of the residue showed the conversion to be 97.5%, a peak at RT 16.2 min (58 area %) consisting of (2S,3S,11bS)- and (2R,3S,11bS)-N-Boc Ethyl ester, a peak at RT 18.2 min (4.1 area %) consisting of (2S,3S,11bS)-N-Boc-Trifluoromethyl ester, a peak at RT 17.4 min (4.6 area %) consisting of (2R,3R,11bS)-N-Boc-Ester and a peak at RT 20.3 min (3.6 area %) consisting of (2S,3R,11bS)-N-Boc-Ester.

### c) Amidation

The carboxylic ester group was converted into the corresponding amide by treatment of the residue in THF with formamide and sodium methylate solution in an analogous manner as described in Example 1. HPLC analysis showed the mixture to contain 44% of the desired (2S,3S,11bS)-N-Boc-Amide 8.

### Examples 3.1 to 3.5

The following experiments in Table 1 below have been carried out in analogy to example 2 using various non-chiral diphosphines for the in-situ formation of the catalyst with [Rh(COD)TFA]₂, S/C 25.

TABLE 1

| | | | Content of |
|---|---|---|---|
| Exam- | | Conver- | (all-S)-N—Boc- |
| ple | Diphosphine | sion (%) | amide ^{a)} (%) |
| 3.1 | DPPP | 36 | 21.7 |
| 3.2 | DPPB | 71 | 57 |
| 3.3 | DiPPB | 99.6 | 26 |
| 3.4 | 1,2-Bis(iPr2P)- | 98 | 62 |
| | acenaphthylene | | |
| 3.5 | DiPPP | 99 | 33 |

^{a)} Determined by HPLC after amidation reaction with formamide and sodium methylate solution, area %.

### Example 4

The experiments in Table 2 have been carried out in analogy to example 2 using various chiral diphosphines for the in-situ formation of the catalyst with [Rh(COD)TFA]₂ (precursor A), [Rh(COD)Cl]₂ (precursor B) or [Rh(COD)2]OTf (precursor C), S/C 25.

TABLE 2

| | | | | Content of |
|---|---|---|---|---|
| | | | Conver- | (all-S)- |
| Exam- | | Precur- | sion | N—Boc- |
| ple | Diphosphine | sor | (%) | amide^{a)} (%) |
| 4.1 | (R,S)-PPF—P(tBu)2 | B | 99.6 | 14 ^{b)} |
| 4.2 | (S,R)-PPF—P(tBu)2 | B | 100 | 79 ^{c)} |
| 4.3 | (R)- | C | 95 | 42 |
| | CyMeOBIPHEP | | | |
| 4.4 | (S)- | C | 95 | 34 |
| | CyMeOBIPHEP | | | |
| 4.5 | (R,R)-MeDuphos | C | 99.3 | 13 |
| 4.6 | (S,S)-MeDuphos | C | 99.2 | 36 |
| 4.7 | (R,R)- | A | 93 | 63 |
| | SKEWPHOS | | | |
| 4.8 | (S,S)- | A | 92 | 42 |
| | SKEWPHOS | | | |

^{a)}Determined by HPLC after amidation reaction with formamide and sodium methylate solution, area %;
^{b)} Experiment carried out on 0.5 g of (S)-Enamine ethyl ester as substrate in analogy to example 1;
^{c)} 0.60 g of (S)-Enamine ethyl ester was used as substrate in a 35 ml autoclave at S/C 25, isolated yield of (all-S)-N—Boc-amide was 70%.

### Example 5

The experiments in Tables 3a and 3b have been carried out in analogy to example 2 using various chiral diphosphines for the in-situ formation of the catalyst with [Rh(COD)TFA]₂ (precursor A), [Rh(COD)Cl]₂ (precursor B) or [Rh(COD)₂]OTf (precursor C), [Rh(COD)₂]SbF₆ (precursor D), S/C 25.

TABLE 3a

| | | | | Content of |
|---|---|---|---|---|
| | | | Conver- | (all-S)- |
| Exam- | | Precur- | sion | N—Boc- |
| ple | Diphosphine | sor | (%) | amide^{a)} (%) |
| 5.1 | (1R,1′R,2S,2′S)- | A | 98 | 51 ^{b)} |
| | DuanPhos (163) | | | |
| 5.2 | (S,S)-BCPM (194) | A | 99 | 73 |
| 5.3 | (R,R)-(Cy₂)(3,5-tBu)2- | A | >99 | 71 |
| | DIOP (228) | | | |
| 5.4 | (R)-Cy₂-BIPHEMP | A | >99 | 71 |
| | (136) | | | |
| 5.5 | (S)-Binapine (158) | A | 99 | 56 |
| 5.6 | (S,S,R)-MePHOS- | A | 93 | 45 |
| | MeOBIPHEP (188) | | | |
| 5.7 | (R)-iPr—MeOBIPHEP | A | 84 | 34 |
| | (189) | | | |
| 5.8 | (R)-Et₂-BIPHEMP | A | 99 | 62 |
| | (236) | | | |
| 5.9 | (R,R)- | A | >99 | 27 |
| | Xyl₂PPhFcCHCH₃—PXyl₂ | | | |
| | (214) | | | |
| 5.10 | (R,R)- | A | >99 | 47 |
| | Ph₂PPhFCCHCH₃PPh₂ | | | |
| | (231) | | | |
| 5.11 | (R,R)- | A | >99 | 46 |
| | Ph₂PPhFcCHCH₃PXyl₂ | | | |
| | (233) | | | |
| 5.12 | (S,S)-Ph-BPE (342) | C | >99 | 74 |
| 5.13 | (R,S,S)-(Cy,Ph)₂- | C | 88 | 66 |
| | BIPHEMP | | | |
| 5.14 | (R)-(Cy)₂(pTolyl)₂- | C | >99 | 82 |
| | BIPHEMP | | | |

TABLE 3b

| | | | | Content of |
|---|---|---|---|---|
| | | | Conver- | (all-S)- |
| Exam- | | Precur- | sion | N—Boc- |
| ple | Diphosphine | sor | (%) | amide^{a)} (%) |
| 5.15 | (R,R)-PPF—PCy₂ (105) | D | 98 | 54 |
| 5.16 | (R,R)-PPF—PCy₂ (117) | A | 99 | 59 |
| 5.17 | (S,R)-Cy₂PF—PPh₂ (195) | A | >99 | 49 |
| 5.18 | (R,S)-Cy-PPF—P(Cy)₂ | A | >99 | 34 |
| | (225) | | | |
| 5.19 | (S,R)-PPF—PCy₂ | D | >99 | 67 |
| 5.20 | (S,R)-PPF—CH₂P(tBu)₂ | C | >99 | 80 |
| 5.21 | (S,R)-Furyl₂PF—P(tBu)₂ | D | >99 | 76 |
| 5.22 | (R)-PP(4- | C | 98 | 78 |
| | Ph)F—CH₂P(tBu)₂ | | | |
| 5.23 | (S,R)-(3,5-tBu₂-4- | C | >99 | 75 |
| | MeOPh)₂PF—P(tBu)₂ | | | |
| 5.24 | (S,R)-(2- | C | >99 | 58 |
| | MeOPh)₂PF—P(tBu)₂ | | | |
| 5.25 | (S,R)-(4-F- | C | >99 | 82 |
| | Ph)₂PF—P(tBu)₂ | | | |
| 5.26 ^{c)} | (S,R)-MOD-PPF—P(tBu)₂ | C | 91 | 61 ^{d)} |

^{a)}Determined by HPLC after amidation reaction with formamide and sodium methylate solution, area %;
b) 0.70 g of (S)-Enamine was used as substrate in a 35 ml autoclave at S/C 50;
^{c)} This experiment was carried out at S/C 1500 in analogy to Example 11.
^{d)} Content of (all-S)-N—Boc-Ethyl ester + (2R,3S,11bS)-N—Boc-Ethyl ester + (2S,3S,11bS)-N—Boc-2,2,2-Trifluoroethyl ester (%), not of (all-S)-N—Boc-amide.

### Example 5a

The experiments in Table 4 have been carried out in analogy to example 2 using 50 mg of (S)-Enamine ethyl ester, with [Rh(COD)₂]OTf/(S,R)—PPF—P(tBu)₂ as catalyst at S/C 50 in 1 ml of total solvent.

TABLE 4

| Exam- | Solvent | | Content of |
|---|---|---|---|
| ple | 4:1 vol/vol | Conversion (%) | esters ^{a)} (%) |
| 5a.1 | TFE/MeOH | >99 | 91 ^{b)} |
| 5a.2 | TFE/THF | >99 | 91 |
| 5a.3 | TFE/CH₂Cl₂ | >99 | 83 |
| 5a.4 | TFE/toluene | >99 | 88 |
| 5a.5 | TFE/ethyl acetate | >99 | 91 |
| 5a.6 | TFE/acetone | >99 | 73 |

^{a)} Esters added together: (all-S)-N—Boc-Ethyl ester + (2R,3S,11bS)-N—Boc-Ethyl ester + (2S,3S,11bS)-N—Boc-2,2,2-Trifluoroethyl ester; determined by HPLC after treatment with 50 mg of di-tert.-butyl-dicarbonate, area %.
^{b)} As a mixture of trifluoroethyl and methyl ester.

### Example 5b

The experiments in Table 5 have been carried out in analogy to example 8 under addition of an additive (0.15 mmol).

TABLE 5

| Exam- | | | Content of (all-S)- |
|---|---|---|---|
| ple | Base | Conversion (%) | N—Boc-amide^{a)} (%) |
| 5b.1 | Ammonium | >99 | 71 |
| | acetate | | |
| 5b.2 | Cesium carbonate | >99 | 71 |
| 5b.3 | Sodium formiate | >99 | 88 |
| 5b.4 | Dicyclohexyl | >99 | 83 |
| | amine | | |
| 5b.5 | Diisopropyl | >99 | 82 |
| | ethylamine | | |
| 5b.6 | Triethyl amine | >99 | 83 |

^{a)}Determined by HPLC after amidation reaction with formamide and sodium methylate solution, area %;

### Example 6

### a) In-Situ Preparation of the Catalyst Solution

In a glove box (O₂ content<2 ppm) an Erlenmeyer flask was charged with 7.4 mg [Rh(COD)TFA]₂ (0.011 mmol), 14.0 mg (R)-Cy2-BIPHEMP (0.025 mmol) and 5 mL trifluoroethanol. The mixture was stirred for 2 h at room temperature.

### b) Asymmetric Hydrogenation (S/C 200)

In the glove box 1 ml of the above catalyst solution was added in a glass vial to a solution of 0.30 g (0.90 mmol) of (S)-Enamine ethyl ester 7 in 2 ml of trifluoroethanol and the vial was placed in an autoclave. The autoclave was sealed and pressurized with hydrogen (30 bar). The reaction mixture was hydrogenated during 18 h at 50° C. under stirring. The hydrogenation mixture was removed from the autoclave, 0.306 g (1.4 mmol) of di-tert.-butyl-dicarbonate were added, the mixture was stirred at 40° C. for 1 h and evaporated to dryness in vacuo. HPLC analysis of the residue showed the conversion to be 99.6% with following composition: (2S,3S,11bS)- and (2R,3S,11bS)-N-Boc-Ethyl ester (84 area %), (2S,3S,11bS)-N-Boc-2-Trifluoroethyl ester (7.6 area %), (2R,3R,11bS)-N-Boc-Ester (0.3 area %).

### c) Amidation

The carboxylic ester group was converted into the corresponding amide by treatment of the residue in THF with formamide and sodium methylate solution in an analogous manner as described in Example 1c. HPLC analysis showed the mixture to contain 84% of the desired (2S,3S,11bS)-N-Boc-Amide 8.

### Example 7

### a) In-Situ Preparation of the Catalyst Solution

In a glove box (O₂ content<2 ppm) an Erlenmeyer flask was charged with 7.4 mg [Rh(COD)TFA]₂ (0.011 mmol), 14.8 mg (R)-Cy₂-MeOBIPHEP (0.025 mmol) and 5 mL trifluoroethanol. The mixture was stirred for 2 h at room temperature.

### b) Asymmetric Hydrogenation (S/C 200)

In the glove box 1 ml of the above catalyst solution was added in a glass vial to a solution of 0.30 g (0.90 mmol) of (S)-Enamine ethyl ester 7 in 2 ml of trifluoroethanol and the vial was placed in an autoclave. The autoclave was sealed and pressurized with hydrogen (30 bar). The reaction mixture was hydrogenated during 18 h at 50° C. under stirring. The hydrogenation mixture was removed from the autoclave, 0.306 g (1.4 mmol) of di-tert.-butyl-dicarbonate were added, the mixture was stirred at 40° C. for 1 h and evaporated to dryness in vacuo. HPLC analysis of the residue showed the conversion to be 99.5% with following composition: (2S,3S,11bS)- and (2R,3S,11bS)-N-Boc-Ethyl ester (80 area %), (2S,3S,11bS)-N-Boc-2-Trifluoroethyl ester (6.7 area %), (2R,3R,11bS)-N-Boc-Ester (0.3 area %).

### c) Amidation

The carboxylic ester group was converted into the corresponding amide by treatment of the residue in THF with formamide and sodium methylate solution in an analogous manner as described in Example 1c. HPLC analysis showed the mixture to contain 79% of the desired (2S,3S,11bS)-N-Boc-Amide 8.

### Example 8

### a) In-Situ Preparation of the Catalyst Solution

In a glove box (O₂ content<2 ppm) an Erlenmeyer flask was charged with 7.0 mg [Rh(COD)₂]OTf (0.015 mmol), 9.00 mg (S,R)—PPF—P(tBu)₂ (0.016 mmol) and 5 mL trifluoroethanol. The mixture was stirred for 1.5 h at room temperature.

### b) Asymmetric Hydrogenation (S/C 500)

In the glove box a 35 ml glass-lined autoclave equipped with a magnetic stirring bar was charged with 0.50 g (1.50 mmol) of (S)-Enamine ethyl ester 7, 3 ml of trifluoroethanol and 1 ml of the above catalyst solution. The autoclave was sealed and pressurized with hydrogen (30 bar). The reaction mixture was hydrogenated during 18 h at 50° C. under stirring. The hydrogenation mixture, an orange solution, was removed from the autoclave, 0.492 mg (2.26 mmol) of di-tert.-butyl-dicarbonate were added, the mixture was stirred at 40° C. for 1 h and evaporated to dryness in vacuo. HPLC analysis of the residue showed the conversion to be 99.9% with following composition: (2S,3S,11bS)- and (2R,3S,11bS)-N-Boc-Ethyl ester (77 area %), (2S,3S,11bS)-N-Boc-2-Trifluoroethyl ester (15 area %), (2S,3R,11bS)-N-Boc-Ester (1.9 area %).

### Example 9

### a) In-Situ Preparation of the Catalyst Solution: Same as in Example 8

### b) Asymmetric Hydrogenation (S/C 500)

In the glove box a 35 ml glass-lined autoclave equipped with a magnetic stirring bar was charged with 0.50 g (1.50 mmol) of (S)-Enamine ethyl ester 7, 3 ml of trifluoroethanol and 1 ml of the above catalyst solution. The autoclave was sealed and pressurized with hydrogen (10 bar). The reaction mixture was hydrogenated during 18 h at 50° C. under stirring. The hydrogenation mixture, an orange solution, was removed from the autoclave, 0.492 mg (2.26 mmol) of di-tert.-butyl-dicarbonate were added, the mixture was stirred at 40° C. for 1 h and evaporated to dryness in vacuo. HPLC analysis of the residue showed the conversion to be complete with following composition: (2S,3S,11bS)- and (2R,3S,11bS)-N-Boc-Ethyl ester (77 area %), (2S,3S,11bS)-N-Boc-2-Trifluoroethyl ester (15 area %), (2S,3R,11bS)-N-Boc-Ester (1.3 area %).

### Example 10

### a) In-Situ Preparation of the Catalyst Solution: Same as in Example AH8

### b) Asymmetric Hydrogenation (S/C 500)

In the glove box a 35 ml glass-lined autoclave equipped with a magnetic stirring bar was charged with 0.50 g (1.50 mmol) of (S)-Enamine ethyl ester 7, 3 ml of trifluoroethanol and 1 ml of the above catalyst solution. The autoclave was sealed and pressurized with hydrogen (30 bar). The reaction mixture was hydrogenated during 18 h at 80° C. under stirring. The hydrogenation mixture, an orange solution, was removed from the autoclave, 0.492 mg (226 mmol) of di-tert.-butyl-dicarbonate were added, the mixture was stirred at 40° C. for 1 h and evaporated to dryness in vacuo. HPLC analysis of the residue showed the conversion to be 99.9% with following composition: (2S,3S,11bS)- and (2R,3S,11bS)-N-Boc-Ethyl ester (85 area %), (2S,3S,11bS)-N-Boc-2-Trifluoroethyl ester (9 area %), (2S,3R,11bS)-N-Boc-Ester (1.4 area %).

### c) Amidation

The residue from this example was combined with the residues of examples 8 and 9 and converted to the corresponding amide by treatment with formamide and a 30% solution of sodium methylate in methanol in analogy to example 1c. After filtration and drying of the precipitate 1.46 g (80%) of (S,S,S)—N-Boc-Amide with 98.3 area % purity by HPLC were isolated.

### Example 11

### a) In-Situ Preparation of the Catalyst Solution

In a glove box (O₂ content<2 ppm) an Erlenmeyer flask was charged with 6.9 mg [Rh(COD)₂]OTf (0.015 mmol), 8.15 mg (S,R)—PPF—P(tBu)₂ (0.016 mmol) and 6 mL trifluoroethanol. The mixture was stirred for 2 h at room temperature.

### b) Asymmetric Hydrogenation (S/C 2000)

In the glove box a 185 ml autoclave was charged with 9.97 g (30 mmol) of (S)-Enamine ethyl ester 7, 65 ml of trifluoroethanol and the above catalyst solution. The autoclave was sealed and the hydrogenation was run under stirring under 30 bar of hydrogen at 60° C. After 16 h the autoclave was opened and the reaction mixture, an orange solution, was transferred to a glass flask with aid of 10 ml of tetrahydrofuran. After addition of 9.64 g (44.2 mmol) of di-tert.-butyl-dicarbonate the mixture was stirred at 40° C. for 1.5 h and evaporated to dryness in vacuo. HPLC analysis of the residue showed the conversion to be 99.2% with following composition: (2S,3S,11bS)- and (2R,3S,11bS)-N-Boc-Ethyl ester (80 area %), (2S,3S,11bS)-N-Boc-2,2,2-Trifluoroethyl ester (12 area %), (2S,3R,11bS)-N-Boc-Ester (1.2 area %).

### c) Amidation

The residue was dissolved in 120 ml of tetrahydrofuran and converted to the corresponding amide by treatment with formamide (12 ml, 302 mmol) and a 30% solution of sodium methylate in methanol (16.5 ml, 88.9 mmol) at 36° C. over night. The resulting suspension was treated with water at reflux, cooled to room temperature and filtered with suction. The filter cake was washed thoroughly with a total of 12 ml of THF/water 2:1 mixture. After drying of the precipitate 9.79 g (82%) of (S,S,S)—N-Boc-Amide with 99.6 area % purity by HPLC were isolated.

Elemental Analysis for C₂₁H₃1N₃O₂

| | Calc | found |
|---|---|---|
| C | 62.20 | 61.95 |
| H | 7.71 | 7.61 |
| N | 10.36 | 10.19 |

Residue <0.1%

### Example 12

### a) Preparation of Substrate Solution

In a 250 ml round-bottomed flask a mixture of 20.72 g of (S)-2-amino-9,10-dimethoxy-1,6,7,11b-tetrahydro-4H-pyrido[2,1-a]isoquinoline-3-carboxylic acid ethylester, (2S,3S)-bis-benzoyloxy-succinic acid salt 6, 7.0 g of sodium carbonate, 100 ml of isopropyl acetate and 80 ml of deionized water were stirred vigorously during 30 min. After separation of the aqueous phase, the organic phase was washed with water, dried over sodium sulphate and partially evaporated at the rotavapor to a total weight of 16 g. Theoretical content of (S)-Enamine ethyl ester 7 was 9.97 g. The solution was introduced into the glove-box.

### b) In-Situ Preparation of the Catalyst Solution

In a glove box (O₂ content<2 ppm) an Erlenmeyer flask was charged with 9.37 mg [Rh(COD)₂]OTf (0.02 mmol), 9.37 mg (S,R)—PPF—P(tBu)₂ (0.02 mmol) and 4 mL trifluoroethanol. The mixture was stirred for 2 h at room temperature.

### c) Asymmetric Hydrogenation (S/C 1500)

In the glove box a 185 ml autoclave was charged with the above solution of (S)-Enamine ethyl ester 7, 54 ml of trifluoroethanol and the above catalyst solution.

The autoclave was sealed and the hydrogenation was run under stirring under 30 bar of hydrogen at 60° C. After 16 h the autoclave was opened and the reaction mixture, an orange solution, was transferred to a glass flask with aid of a total of 10 ml of methanol. After addition of 9.82 g (45 mmol) of di-tert.-butyl-dicarbonate the mixture was stirred at 40° C. for 1.5 h and evaporated in vacuo under simultaneous addition of a total of 150 ml of methanol. Finally, the residue (35 g tot) was taken up in 30 ml of tetrahydrofuran. HPLC analysis of the residue showed the conversion to be 97.7% with following composition: (2S,3S,11bS)- and (2R,3S,11bS)-N-Boc-Ethyl ester (77 area %), (2S,3S,11bS)-N-Boc-2,2,2-Trifluoroethyl ester (11.1 area %), (2S,3R,11bS)-N-Boc-Ester (0.3 area %).

### d) Amidation

The above solution was converted to the corresponding amide as described in example 11 by treatment with formamide (12 ml, 302 mmol) and a 30% solution of sodium methylate in methanol (17 ml, 88.9 mmol) at 36° C. over night. After drying of the precipitate 10.11 g (83%) of (S,S,S)—N-Boc-Amide 8 with 98.8 area % purity by HPLC were isolated.

### Example 13

### a) In-Situ Preparation of the Catalyst Solution was Carried Out as in Example 11

In a glove box (O₂ content<2 ppm) an Erlenmeyer flask was charged with 6.9 mg [Rh(COD)2]OTf (0.015 mmol), 8.15 mg (S,R)—PPF—P(tBu)2 (0.016 mmol) and 6 mL trifluoroethanol. The mixture was stirred for 2 h at room temperature.

### b) Asymmetric Hydrogenation (S/C 2000)

In the glove box a 185 ml autoclave was charged with 9.97 g (29 mmol, 96.7% pure) of (S)-Enamine ethyl ester 7, 204 mg (3.0 mmol) of sodium formiate, 60 ml of trifluoroethanol and the above catalyst solution. The autoclave was sealed and the hydrogenation was run under stirring under 30 bar of hydrogen at 60° C. After 16 h the autoclave was opened and the reaction mixture, an orange solution, was transferred to a glass flask with aid of 10 ml of methanol After addition of 9.82 g (45 mmol) of di-tert.-butyl-dicarbonate the mixture was stirred at 40° C. for 1.5 h and evaporated in vacua under continuous addition of 150 ml of methanol to a solution with a total weight of 36 g. HPLC analysis of the residue showed the conversion to be 99.6% with following composition: (2S,3S,11bS)- and (2R,3S,11bS)—N-Boc-Ethyl ester (79 area %), (2S,3S,11bS)—N-Boc-2,2,2-Trifluoroethyl ester (8.6 area %), (2S,3R,11bS)—N-Boc-Ester (0.5 area %).

### c) Amidation

To the above solution were added 100 ml of tetrahydrofuran, then the conversion to the corresponding amide was carried out by treatment with formamide (12 ml, 302 mmol) and a 30% solution of sodium methylate in methanol (17 ml, 91.6 mmol) at 36° C. over night. The resulting suspension was treated with water at reflux, cooled to room temperature and filtered with suction. The filter cake was washed thoroughly with a total of 12 ml of THF/water 2:1 mixture. After drying of the precipitate 9.37 g (80%) of (S,S,S)—N-Boc-Amide 8 with 99.4 area % purity by HPLC were isolated.

### Example 14

### a) In-Situ Preparation of the Catalyst Solution

In a glove box (O₂ content<2 ppm) an Erlenmeyer flask was charged with 7.1 mg [Rh(COD)₂]OTf (0.015 mmol), 8.99 mg (S,R)—PPF—P(tBu)₂ (0.016 mmol) and 5 mL trifluoroethanol. The mixture was stirred for 1 h at room temperature.

### b) Asymmetric Hydrogenation (S/C 1500)

In the glove box a 60 ml autoclave was charged with 1.50 g (4.51 mmol) of (S)-Enamine ethyl ester, 12 ml of trifluoroethanol and 1 ml of the above catalyst solution. The autoclave was sealed and the hydrogenation was run under stirring under 10 bar of hydrogen at 70° C. whereas a solution of 1.50 g of Boc₂O (6.78 mmol) in 7 ml of trifluoroethanol was added by a pump during 4.5 h. After 22 h the autoclave was opened and the reaction mixture, an orange solution, was transferred to a glass flask with aid of a total of 5 ml of methanol. HPLC analysis showed that the ratio of N-Boc-protected to free esters was 1:2.7. After addition of 1.5 g of Boc₂O the mixture was stirred at 40° C. for 1.5 h and evaporated in vacuo. Finally, the residue was taken up in 10 ml of tetrahydrofuran. HPLC analysis of the residue showed the conversion to be 99.8% with following composition: (2S,3S,11bS)- and (2R,3S,11bS)—N-Boc-Ethyl ester (67 area %), (2S,3S,11bS)—N-Boc-2,2,2-Trifluoroethyl ester (22.5 area %), (2S,3R,1 lbS)—N-Boc-Ester (0.8 area %).

### Example 15

The experiments in Table 6 have been carried out in analogy to example 2 using 50 mg (0.15 mmol) of (S)-Ester as substrate and various chiral ruthenium catalysts (0.0066 mmol) (S/C 25).

TABLE 6

| | | | Content of |
|---|---|---|---|
| Exam- | | Conversion | (all-S)-N— |
| ple | Catalyst | (%) | Boc-amide^{a)} (%) |
| 14.1 | (R,S)-PPF—P(tBu)₂/ | >99 | 12 ^{b)} |
| | [Ru(OAc)₂(COD)] | | |
| 14.2 | (S,R)-PPF—P(tBu)₂/ | 99 | 71 ^{b)} |
| | [Ru(OAc)₂(COD)] | | |
| 14.3 | [Ru(OAc)₂((S,S)- | >99 | 63 |
| | SKEWPHOS)] | | |
| 14.4 | [Ru(OAc)₂((all-S)-BICP)] | >99 | 71 |
| 14.5 | [Ru(OAc)₂((S)-TMBTP)] | >99 | 54 |

^{a)}Determined by HPLC after amidation reaction with formamide and sodium methylate solution, area %;
^{b)} The catalyst was prepared in the glove-box in situ by reaction of the chiral diphosphine with [Ru(OAc)₂(COD)] in trifluoroethanol for 2.5 h at room temperature.

### Example 16

### Preparation of (2S,3S,11bS)-(3-Amino-9,10-dimethoxy-1,3,4,6,7,11b-hexahydro-2H-pyrido[2,1-a]isoquinolin-2-yl)]-carbamic acid tert-butyl ester

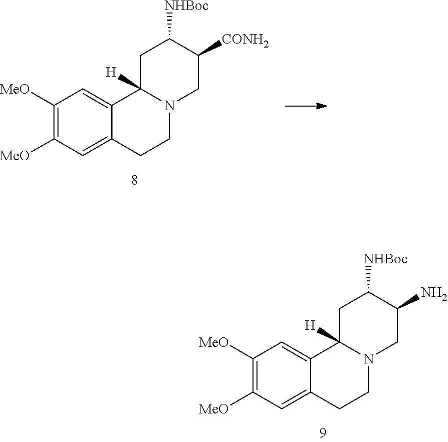 [SMILES: COc1cc2c(cc1OC)[C@@H]1C[C@H](NC(=O)OC(C)(C)C)[C@@H](C(N)=O)CN1CC2 ; COc1cc2c(cc1OC)[C@@H]1C[C@H](NC(=O)OC(C)(C)C)[C@@H](N)CN1CC2]

A 6 L four-necked flask equipped with a mechanical stirrer, a Pt-100 thermometer, a dropping funnel and a nitrogen inlet was charged with 100 g (242 mmol) amide 7 982 ml 2 N sodium hydroxide solution were added and the mixture stirred for 5 minutes at RT. 1.75 L acetonitrile were added and stirring was continued for an additional 30 min. To the resulting suspension was added a solution of 95.5 g (291 mmol) diacetoxyiodosobenzene in 240 ml water and 500 ml acetonitrile during 15 min, maintaining the temperature at 18-22° C. The slightly yellow reaction mixture was stirred at RT for 15 min. A slightly yellow two-phase mixture containing some undissolved crystals was formed, to which 400 g sodium chloride were added and the mixture was further stirred for 20 minutes at RT, then cooled to 5° C. A solution of 220 ml 25% hydrochloric acid and 220 ml water were slowly added during 30 min to bring the pH to about 5.5. From pH of 8 on, a precipitate formed. The suspension was further stirred for 75 minutes at 5 to 10° C. and pH 5.5. The suspension was filtered off, transferred back into the reactor and suspended in 1.5 L dichloromethane. 1 L of a 10% sodium bicarbonate solution was added to the suspension and the mixture was stirred for 15 minutes, whereas pH 8 was reached. The organic phase was separated and the aqueous phase was extracted again with 1 L dichloromethane. The organic phases were collected and concentrated at 45° C. to just before the crystallization point. 275 ml TBME were added and the resulting suspension stirred for 1 hour at RT and then for 1.5 hour at 0 to 4° C. The crystals were then filtered off and washed portionwise with totally 150 ml of cold TBME.

The crystals were dried at 40-45° C. at 10 mbar for 48 hours, then suspended in a mixture of 530 ml ethanol and 530 ml methanol and stirred for 2 hours at RT. The precipitate was filtered off and washed portionwise with totally 100 ml of a 1:1 mixture of methanol and ethanol. The filtrate was evaporated to dryness at 50° C. and the crystals dried at 50° C./1 mbar. They were then suspended in 400 ml TBME, stirred for 2 hours at 20° C. and then for 2 hours at 0° C. The crystals were filtered off and washed portionwise with totally 200 ml cold TBME. The crystals were dried at 40-45° C. at ≦20 mbar for 24 hours to give 67.2 g amine 9 (73% yield; assay: 99%)

### Example 17

Transformation of (2S,3S,11bS)-(3-Amino-9,10-dimethoxy-1,3,4,6,7,11b-hexahydro-2H-pyrido[2,1-a]isoquinolin-2-yl)]-carbamic acid tert-butyl ester into (S)-1-(2S,3S,11bS)-2-amino-9,10-dimethoxy-1,3,4,6,7,11b-hexahydro-2H-pyrido[2,1-a]isoquinolin-3-yl)-4-fluoromethyl-pyrrolidin-2-one

### a) Preparation of 4-fluoromethyl-5H-furan-2-one

A 6 L reactor equipped with a mechanical stirrer, a Pt-100 thermometer, a dropping funnel and a nitrogen inlet was charged with 500 g (4.38 mmol) 4-hydroxymethyl-5H-furan-2-one and 2.0 L dichloromethane. The solution was cooled to −10° C. and 1.12 kg (4.82 mol) bis-(2-methoxyethyl)aminosulfur trifluoride (Deoxo-Fluor) was added during 50 min, maintaining the temperature at −5 to −10° C. with a cooling bath. During the addition a yellowish emulsion formed, which dissolved to an orange-red solution after completed addition. This solution was stirred for 1.5 h at 15-20° C., then cooled to −10° C. A solution of 250 ml water in 1.00 L ethanol was added during 30 min, maintaining the temperature between −5 and −10° C., before the mixture was allowed to reach 15-20° C. It was then concentrated in a rotatory evaporator to a volume of ca. 1.6 L at 40° C./600-120 mbar. The residue was dissolved in 2.0 L dichloromethane and washed three times with 4.0 L 1N hydrochloric acid. The combined aqueous layers were extracted three times with 1.4 L dichloromethane. The combined organic layers were evaporated in a rotatory evaporator to give 681 g crude product as a dark brown liquid. This material was distilled over a Vigreux column at 0.1 mbar, the product fractions being collected between 71 and 75° C. (312 g). This material was re-distilled under the same conditions, the fractions being collected between 65 and 73° C., to give 299 g 4-fluoromethyl-5H-furan-2-one (58% yield; assay: 99%).

MS: m/e 118 M⁺, 74, 59, 41

### b) Preparation of (S)-4-fluoromethyl-dihydro-furan-2-one

A 2 L autoclave equipped with a mechanical stirrer was charged with a solution of 96.0 g 4-fluoromethyl-5H-furan-2-one (8.27×10-1 mol) in 284 mL methanol. The autoclave was sealed and pressurized several times with argon (7 bar) in order to remove any traces of oxygen. At ˜1 bar argon, a solution of 82.74 mg Ru(OAc)₂((R)-3,5-tBu-MeOBIPHEP) (6.62×10-5 mol) (S/C 12500) in 100 mL methanol was added under stirring from a catalyst addition device previously charged in a glove box (O₂ content<2 ppm) and pressurized with argon (7 bar). The argon atmosphere in the autoclave was replaced by hydrogen (5 bar). At this pressure, the reaction mixture was stirred (˜800 rpm) for 20 h at 30° C. and then removed from the autoclave and concentrated in vacuo. The residue was distilled to afford 91.8 g (94%) (S)-4-fluoromethyl-dihydro-furan-2-one. The chemical purity of the product was 99.7% by GC-area.

### c) Preparation of (2S,3S,11bS)-3-((S)-3-Fluoromethyl-4-hydroxy-butyrylamino)-9,10-dimethoxy-1,3,4,6,7,11b-hexahydro-2H-pyrido[2,1-a]isoquinolin-2-yl]-carbamic acid tert-butyl ester

A 1.5 L reactor equipped with a mechanical stirrer, a Pt-100 thermometer, a dropping funnel and a nitrogen inlet was charged with 50 g (128 mmol) (2S,3S,11bS)-3-amino-9,10-dimethoxy-1,3,4,6,7,11b-hexahydro-2H-pyrido[2,1-a]isoquinolin-2-yl)-carbamic acid tert-butyl ester, 500 mL toluene and 2.51 g (25.6 mmol) 2-hydroxypyridine. To this slightly brownish suspension, 22.7 g (192 mmol) of (S)-4-fluoromethyl-dihydro-furan-2-one was added dropwise at RT. No exothermy was observed during the addition. The dropping funnel was rinsed portionwise with totally 100 mL toluene. The suspension was heated to reflux, whereas it turned into a clear solution starting from 60° C., after 40 min under reflux a suspension formed again. After totally 23 h under reflux, the thick suspension was cooled to RT, diluted with 100 mL dichloromethane and stirred for 30 min at RT. After filtration, the filter cake was washed portionwise with totally 200 mL toluene, then portionwise with totally 100 mL dichloromethane. The filter cake was dried at 50° C./10 mbar for 201, to give 60.0 g product (94% yield; assay: 100%).

MS: m/e 496 (M+H)⁺, 437

### d) Preparation of (2S,3S,11bS)-3-((4S)-Fluoromethyl-2-oxo-pyrrolidin-1-yl)-9,10-dimethoxy-1,3,4,6,7,11b-hexahydro-2H-pyrido[2,1-a]isoquinolin-2-yl]-carbamic acid tert-butyl ester

A 1.5 L reactor equipped with a mechanical stirrer, a Pt-100 thermometer, a dropping funnel, a cooling bath and a nitrogen inlet was charged with 28 g (56.5 mmol) of (2S,3S,11bS)-3-((S)-3-fluoromethyl-4-hydroxy-butyrylamino)-9,10-dimethoxy-1,3,4,6,7,11b-hexahydro-2H-pyrido[2,1-a]isoquinolin-2-yl]-carbamic acid tert-butyl ester and 750 mL THF. The mixture was cooled to 0° C. and a solution of 6.17 mL (79 mmol) methanesulfonic acid in 42 mL THF was added during 10 min, maintaining the temperature at 0-5° C. At 0° C. a solution of 12.6 mL (90.2 mmol) triethylamine in 42 mL THF was added during 15 min. The resulting suspension was stirred for 80 min at 0-5° C., whereas it became gradually thicker. Then 141 ml, (141 mmol) 1 M lithium-bis(trimethylsilyl)amide were added to the mixture during 15 min, whereas the suspension dissolved. The solution was allowed to reach RT during 60 min under stirring. 500 mL water was added without cooling, the mixture was extracted and the aqueous phase was subsequently extracted with 500 mL and 250 mL dichloromethane. The organic layers were each washed with 300 mL half saturated brine, combined and evaporated on a rotatory evaporator. The resulting foam was dissolved in 155 mL dichloromethane, filtered and again evaporated to give 30.5 g crude product as a slightly brownish foam. This material was dissolved in 122 mL methanol, resulting in a thick suspension, which dissolved on heating to reflux. After 20 min of reflux the solution was allowed to gradually cool to RT during 2 h, whereas crystallization started after 10 min. After 2 h the suspension was cooled to 0° C. for 1 h, followed by −25° C. for 1 h. The crystals were filtered off via a pre-cooled glasssinter funnel, washed portionwise with 78 mL TBME and dried for 18 h at 45° C./20 mbar, to give 21.0 g product RO4876706 as white crystals (77% yield; assay: 99.5%).

MS: m/e 478 (M+H)⁺, 437, 422.

### e) Preparation of (2S,3S,11bS)-1-(2-amino-9,10-dimethoxy-1,3,4,6,7,11b-hexahydro-2H-pyrido[2,1-a]isoquinolin-3-yl)-4(S)-fluoromethyl-pyrrolidin-2-one dihydrochloride

A 2.5 L reactor equipped with a mechanical stirrer, a Pt-100 thermometer, a dropping funnel and a nitrogen inlet was charged with 619 g (1.30 mol) of (2S,3S,11 bS)-3-((4S)-fluoromethyl-2-oxo-pyrrolidin-1-yl)-9,10-dimethoxy-1,3,4,6,7,11b-hexahydro-2H-pyrido[2,1-a]isoquinolin-2-yl]carbamic acid tert-butyl ester, 4.2 L isopropanol and 62 mL water and the suspension was heated to 40-45° C. In a second vessel, 1.98 L isopropanol was cooled to 0° C. and 461 ml, (6.50 mol) acetyl chloride was added during 35 min, maintaining the temperature at 0-7° C. After completed addition, the mixture was allowed to reach ca. 15° C. and was then slowly added to the first vessel during 1.5 h. After completed addition the mixture was stirred for 18 h at 40-45° C., whereas crystallization started after 1 h. The white suspension was cooled to 20° C. during 2 h, stirred at that temperature for 1.5 h and filtered. The crystals were washed portionwise with 1.1 L isopropanol and dried for 72 h at 45° C./20 mbar, to give 583 g of the product as white crystals (100% yield; assay: 99.0%).

It is to be understood that the invention is not limited to the particular embodiments of the invention described above, as variations of the particular embodiments may be made and still fall within the scope of the appended claims

What is claimed is:

1. A process for the preparation of pyrido[2,1-a]isoquinoline derivatives of the formula 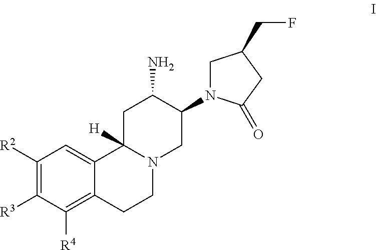
wherein R² and R³ are lower alkoxy and R⁴ is hydrogen,
comprising the steps a) and/or b) and/or c), wherein
step a) comprises catalytic asymmetric hydrogenation of an enamine of the formula 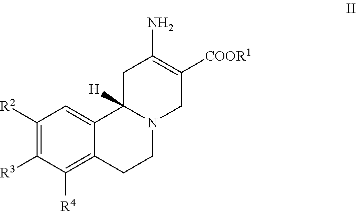
wherein R², R³ and R⁴ are as defined above, wherein further the asymmetric hydrogenation is performed with a rhodium complex containing a chiral diphosphine ligand selected from the group consisting of ((R)-Cy₂-BIPHEMP, (R)-Cy₂-MeOBIPHEP, (S,R)-MOD-PPF—P(tBu)₂ and (S,R)—PPF—P(tBu)₂, and R¹ is lower alkyl, in the presence of a transition metal catalyst to form the (all-S)-amino ester of formula IIIa, alone or as a mixture with 3R-epimer IIIb 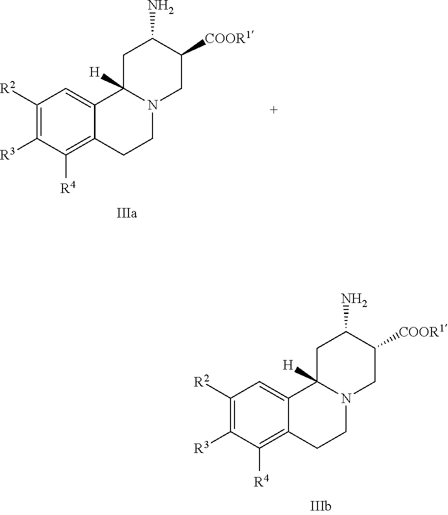
wherein R², R³ and R⁴ are as defined above and R^{1′} is lower alkyl or halogenated lower alkyl;
step b) comprises the introduction of an amino protecting group Prot to form the N-protected (2S)-amino esters of formula 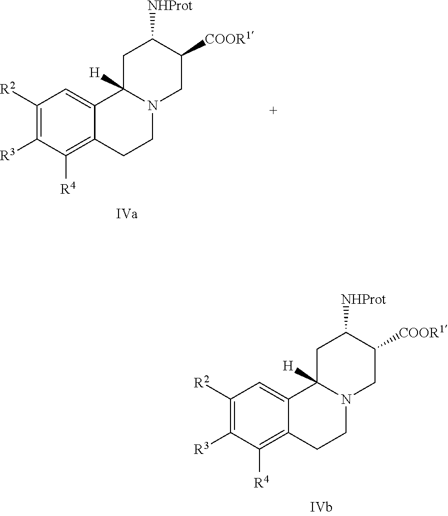
wherein R^{1′}, R², R³ and R⁴ are as defined above and Prot stands for an amino protecting group;
step c) comprises amidation of the ester of formula IV to form the amide of formula 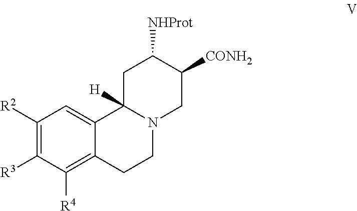
step d) comprises degradation of the amide of formula V to form a carbamic acid ester, followed by coupling of the resulting carbamic acid ester with (S)-4-fluoromethyl-dihydro-furan-2-one of formula VII, 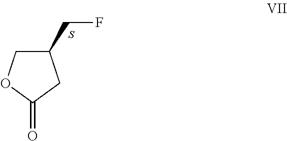
then cyclization in presence of a base and by deprotection of the amino group to form the pyrido[2,1-a] isoquinoline derivatives of formula I
wherein R², R³, R⁴ and Prot are as defined above.

2. The process according to claim 1, characterized in that the asymmetric hydrogenation in step a) is performed with a transition metal catalyst selected from a ruthenium, rhodium or iridium complex catalyst containing a diphosphine ligand.

3. The process according to claim 1, characterized in that the asymmetric hydrogenation in step a) is performed with a rhodium complex catalyst containing a diphosphine ligand.

4. The process according to claim 1, characterized in that the diphosphine ligand is selected from the group consisting of formula A to Q 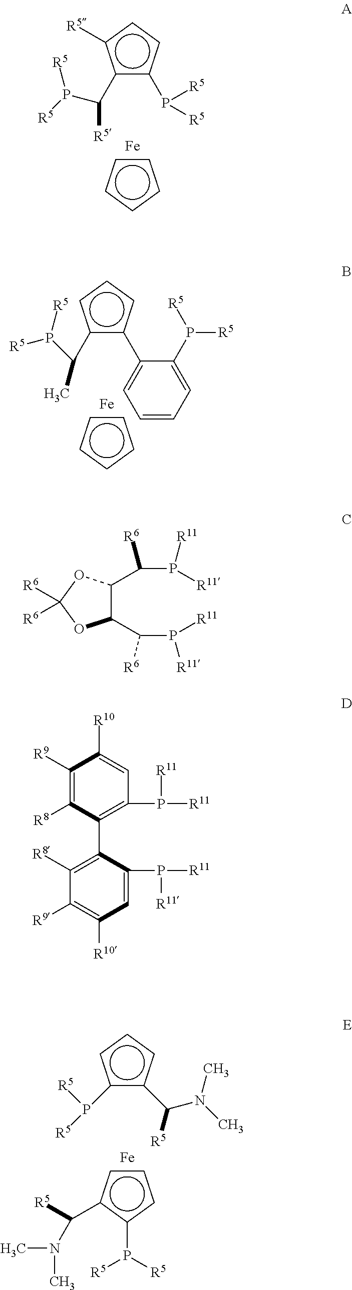 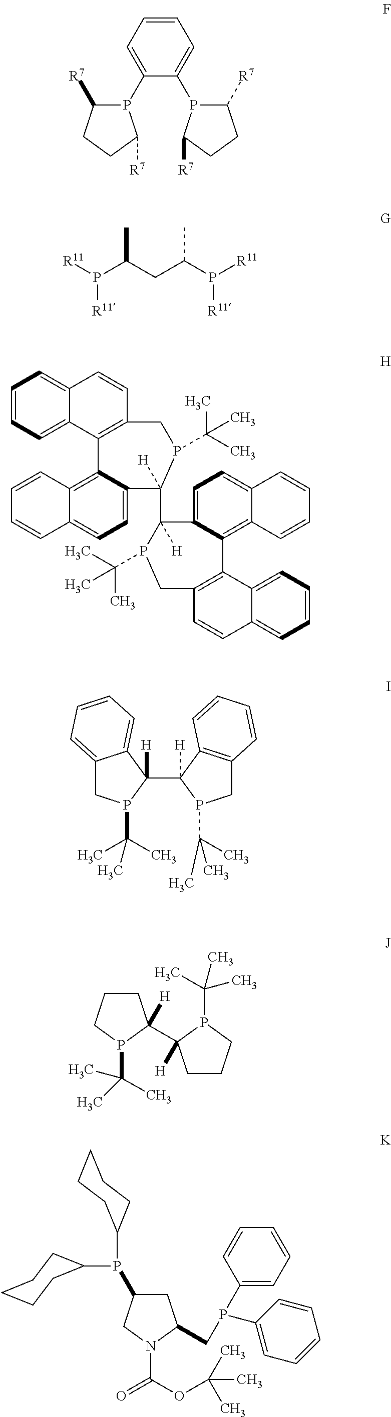 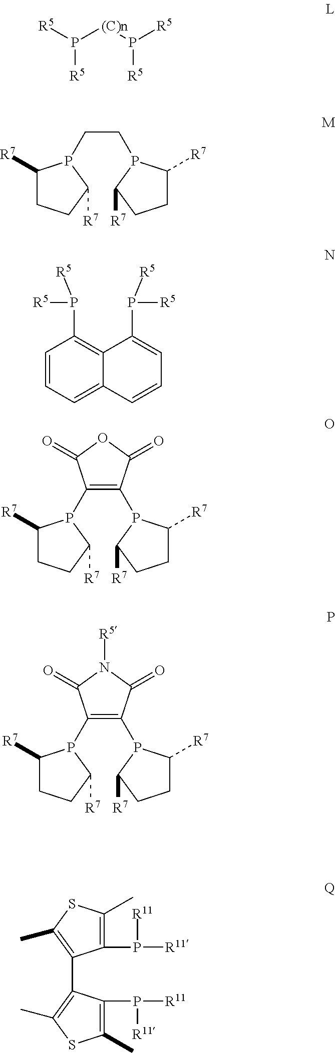
wherein
each R⁵ independently from each other is selected from the group consisting of aryl¹, heteroaryl, cycloalkyl and lower alkyl;
R^{5′} is selected from the group consisting of hydrogen and lower alkyl;
R^{5″} is selected from the group consisting of hydrogen, lower alkyl and phenyl;
each R⁶ independently from each other is lower alkyl;
each R⁷ independently from each other is lower alkyl or aryl¹;
R⁸ and R^{8′} independently from each other are selected from the group consisting of lower alkyl, lower alkoxy, hydroxy and —O—C(O)-lower alkyl;
R⁹, R^{9′}, R¹⁰ and R^{10′} independently from each other are selected from the group consisting of hydrogen, lower alkyl, lower alkoxy and lower dialkylamino; or
R⁸ and R⁹, R^{8′} and R^{9′}, R⁹ and R¹⁰, R^{9′} and R^{10′} or R⁸ and R^{8′}, taken both together, are —X—(CH₂)ₙ—Y—,
wherein X is —O— or —C(O)O—, Y is —O— or —N(lower alkyl)- and n is an integer from 1 to 6; or
R⁸ and R⁹, R^{8′} and R^{9′}, R⁹ and R¹⁰ or R^{9′} and R^{10′}, taken both together, are a —CF₂— group, or together with the carbon atoms to which they are attached, form a naphthyl, tetrahydronaphthyl, dibenzothienyl or dibenzofuranyl ring; and
R¹¹ and R^{11′} independently from each other is selected from the group consisting of aryl¹, lower alkyl, heteroaryl and cycloalkyl; or
R¹¹ and R^{11′} together form a chiral phospholane or phosphetane ring.

5. The process according to claim 1, characterized in that the diphosphine ligand is of the formula 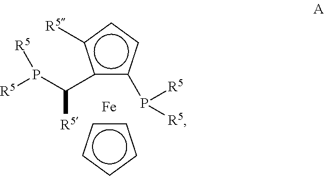
wherein
each R⁵ independently from each other is selected from the group consisting of aryl¹, heteroaryl, cycloalkyl and lower alkyl;
R^{5′} is selected from the group consisting of hydrogen and lower alkyl; and
R^{5″} is selected from the group consisting of hydrogen, lower alkyl and phenyl.

6. The process according to claim 1, characterized in that the asymmetric hydrogenation in step a) is performed with a rhodium complex catalyst containing (S,R)—PPF—P(tBu)₂ as chiral diphosphine ligand.

7. The process according to claim 1, characterized in that the asymmetric hydrogenation is carried out in an inert organic solvent.

8. The process according to claim 8, characterized in that the asymmetric hydrogenation is carried out in 2,2,2-trifluoroethanol.

9. The process according to claim 1, characterized in that the asymmetric hydrogenation takes place at a hydrogen pressure in a range from 1 bar to 200 bar.

10. The process according to claim 1, characterized in that the asymmetric hydrogenation takes place at a reaction temperature in a range from 20° C. to 120° C.

11. The process according to claim 1, characterized in that in step b) tert-butoxycarbonyl is introduced as amino protecting group.

12. The process according to claim 1, characterized in that the amidation in step c) is performed with formamide/sodium methoxide, formamide/sodium ethoxide, acetamide/sodium methoxide and acetamide/sodium ethoxide.

13. The process according to claim 1, characterized in that the amidation in step c) is performed in an organic solvent at temperatures of 10° C. to 70° C.

14. A process for the preparation of (S)-1-((2S,3S,11bS)-2-amino-9,10-dimethoxy-1,3,4,6,7,11b-hexahydro-2H-pyrido[2,1-a]isoquinolin-3-yl)-4-fluoromethyl-pyrrolidin-2-one.

15. The process according to claim 14 for the preparation of (S)-1-((2S,3S,11bS)-2-amino-9,10-dimethoxy-1,3,4,6,7,11b-hexahydro-2H-pyrido[2,1-a]isoquinolin-3-yl)-4-fluoromethyl-pyrrolidin-2-one, comprising the process according to claims 1 to 14, followed by
d) degradation of [(2S,3S,11bS)-(3-Carbamoyl-9,10-dimethoxy-1,3,4,6,7,11b-hexahydro-2H-pyrido[2,1-a]isoquinolin-2-yl)]-carbamic acid tert-butyl ester
e) coupling of the so obtained (2S,3S,11bS)-3-amino-9,10-dimethoxy-1,3,4,6,7,11b-hexahydro-2H-pyrido[2,1-a]isoquinolin-2-yl)-carbamic acid tert-butyl ester with the (S)-4-fluoromethyl-dihydro-furan-2-one of formula 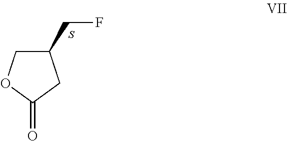
f) cyclization of the obtained (2S,3S,11bS)-3-(3-fluoromethyl-4-hydroxy-butyrylamino)-9,10-dimethoxy-1,3,4,6,7,11b-hexahydro-2H-pyrido[2,1-a] isoquinolin-2-yl]-carbamic acid tert-butyl ester in the presence of a base, and
g) deprotecting the obtained (2S,3S,11bS)-3-((4S)-fluoromethyl-2-oxo-pyrrolidin-1-yl)-9,10-dimethoxy-1,3,4,6,7,11b-hexahydro-2H-pyrido[2,1-a]isoquinolin-2-yl]-carbamic acid tert-butyl ester.
